(19) United States
(12) Patent Application Publication
(10) Pub. No.: US 2021/0292837 A1
(43) Pub. Date: Sep. 23, 2021

(54) ANALYTE DETECTION

(71) Applicants: The Regents of the University of Michigan, Ann Arbor, MI (US); Bio-Rad Laboratories, Inc., Hercules, CA (US)

(72) Inventors: Alexander Johnson-Buck, Ann Arbor, MI (US); Nils Walter, Ann Arbor, MI (US); Muneesh Tewari, Ann Arbor, MI (US); William Bradley Strong, El Cerrito, CA (US); Kenneth J. Oh, Pleasant Hill, CA (US); Evan Thrush, San Anselmo, CA (US); Ning Liu, Walnut Creek, CA (US)

(21) Appl. No.: 17/193,060

(22) Filed: Mar. 5, 2021

Related U.S. Application Data
(60) Provisional application No. 62/991,947, filed on Mar. 19, 2020.

(51) Int. Cl. C12Q 1/6876; C12Q 1/6809; C12Q 1/6834

(52) U.S. Cl. CPC C12Q 1/6876; C12Q 1/6834; C12Q 1/6809

(57) ABSTRACT

Provided herein is technology relating to the detection of analytes and particularly, but not exclusively, to methods, systems, compositions, and kits for detecting analytes such as nucleic acids, proteins, small molecules, metabolites, and other molecules using a technology based on the transient binding of detection probes in combination with a microfluidic device and/or a nanoparticle.

This application claims priority to U.S. provisional patent application Ser. No. 62/991,947, filed Mar. 19, 2020, which is incorporated herein by reference in its entirety.

### STATEMENT REGARDING FEDERALLY SPONSORED RESEARCH OR DEVELOPMENT

This invention was made with government support under CA204560 and CA229023 awarded by the National Institutes of Health. The government has certain rights in the invention.

### FIELD

Provided herein is technology relating to the detection of analytes and particularly, but not exclusively, to methods, systems, compositions, and kits for detecting analytes such as nucleic acids, proteins, small molecules, metabolites, and other molecules using a technology based on the transient binding of detection probes.

### BACKGROUND

Detecting and quantifying low-concentration analytes in complex mixtures has numerous applications in biological research and clinical diagnostics. Many important biological analytes are biomarkers of disease and other biological states. For example, the detection of a small fraction of circulating nucleic acids bearing oncogenic mutations in blood, urine, saliva, and other body fluids has been correlated to the incidence of certain types of cancer. In addition, protein analytes such as prostate-specific antigen (PSA) and interleukins also have current or potential clinical and research significance. Accordingly, the presence and/or levels of analytes provide information about health and drug processing in a biological system. Thus, technologies for detecting and/or quantifying analytes in samples are needed.

### SUMMARY

Accordingly, provided herein is a technology for improving the detection of analytes (e.g., biomolecules (e.g., nucleic acids (e.g., DNA, RNA, methylated and other modified or non-naturally occurring nucleobases), polypeptides (e.g., peptides, proteins, glycoproteins), carbohydrates, lipids, post-translational modifications, amino acids, metabolites, small molecules, etc.) using single-molecule recognition with equilibrium Poisson sampling (SiMREPS) as described in U.S. Pat. No. 10,093,967; U.S. patent application Ser. Nos. 16/154,045; 16/076,853; 15/914,729; 16/219,070; and Int'l Pat. App. No. PCT/US19/43022, each of which is incorporated herein by reference. In some embodiments, the technology relates to using nanoparticles to capture an analyte (e.g., a biomarker) for subsequent analysis by SiMREPS. In some embodiments, the technology relates to SiMREPS using two or more transiently binding query probes that are labeled with two or more different fluorophores and detecting the repeated binding of the multiple probes to an analyte and/or transient Forster resonance energy transfer between the two or more different fluorophores. In some embodiments, the technology relates to increasing the SiMREPS data collection rate by modifying reaction conditions to increase the speed of association and dissociation of query probes and the analyte. In some embodiments, the technology relates to using a microfluidic device to improve analyte capture efficiency and detection of query probe interactions with the analyte. In some embodiments, the technology relates to cross-linking an analyte to a capture probe to prevent dissociation of the analyte from the surface prior to or during measurements. In some embodiments, two or more of these technologies are used in combination (e.g., two or more of using: 1) nanoparticles, 2) two or more query probes, 3) modifying reaction conditions to increases association/dissociation of query probes, 4) a microfluidic device, and/or 5) cross-linking analyte to capture probe). In some embodiments, concentration of analytes at a surface is followed by surface capture of analytes (e.g., immobilization of analytes at the surface). In some embodiments, concentration of analytes at a surface and, optionally, surface capture of analytes at a surface is followed by analysis of the analytes by SiMREPS.

The technology provides advantages over prior technologies including, but not limited to, improved speed of SiMREPS data collection (e.g., lower time-to-result), improved SiMREPS sensitivity, and/or improved SiMREPS specificity.

The technology is not limited in the analyte that is detected. For example, embodiments provide for detection of an analyte that is a nucleic acid, a polypeptide, a carbohydrate, a polysaccharide, a fatty acid, a phospholipid, a glycolipid, a sphingolipid, a small molecule, a metabolite, a cofactor, etc.

In some embodiments, the query and/or capture probe is a nucleic acid or a polypeptide (e.g., an antibody, antibody fragment, linear antibody, single-chain antibody, or other antigen-binding antibody derivative; an enzyme; a binding protein that recognizes the analyte with specificity). In some embodiments in which the analyte comprises a carbohydrate or polysaccharide, the query probe comprises a carbohydrate-binding protein such as a lectin or a carbohydrate-binding antibody. In some embodiments, the presence of a specific glycosidic linkage or set of glycosidic linkages between carbohydrate monomers yields a distinguishable pattern of query probe binding. In some embodiments, the capture probe is a monoclonal antibody; and in some embodiments, the query probe is a mouse or rabbit monoclonal antibody.

In some embodiments, characterizing the analyte comprises indicating the presence, absence, concentration, or number of the analyte in the sample. In some embodiments, the analyte comprises a polypeptide. In some embodiments, the method indicates the presence or absence of a post-translational modification on the polypeptide. In some embodiments, the post-translational modification mediates a transient association of the query probe with the polypeptide. In some embodiments, a chemical affinity tag mediates a transient association between the post-translational modification and the query probe. In some embodiments, the chemical affinity tag is a nucleic acid. In some embodiments, the analyte is a nucleic acid. In some embodiments, a transient association of the query probe with the analyte is distinguishably affected by a covalent modification of the analyte. In some embodiments, the query probe is a nucleic acid or aptamer. In some embodiments, the query probe is a low-affinity antibody, an antibody fragment, or a nanobody. In some embodiments, the query probe is a DNA-binding protein, an RNA-binding protein, or a DNA-binding ribonucleoprotein complex.

In some embodiments, the analyte is subjected to thermal denaturation in the presence of a carrier prior to surface immobilization. In some embodiments, the analyte is subjected to chemical denaturation in the presence of a carrier prior to surface immobilization, e.g., the analyte is denatured with a denaturant such as urea, formamide, guanidinium chloride, high ionic strength, low ionic strength, high pH, low pH, or sodium dodecyl sulfate (SDS).

Further embodiments provide a system for quantifying an analyte in a sample. For example in some embodiments, systems comprise a functionality to stably immobilize an analyte to a surface; a freely diffusing query probe that binds to the analyte with a low affinity; and a detection system that records query probe events and the spatial position of query probe events for analytes. In some embodiments, systems further comprise analytical procedures to identify an individual molecular copy of the analyte according to the spatial position and timing of repeated binding and dissociation events to said analyte. In some embodiments, the query probe is a nucleic acid or aptamer. In some embodiments, the query probe is a low-affinity antibody, an antibody fragment, or a nanobody. In some embodiments of systems, the query probe and/or the capture probe is a DNA-binding protein, an RNA-binding protein, or a DNA-binding ribonucleoprotein complex. In some embodiments, the query and/or capture probe is a nucleic acid or a polypeptide (e.g., an antibody, antibody fragment, linear antibody, single-chain antibody, or other antigen-binding antibody derivative; an enzyme; a binding protein that recognizes the analyte with specificity). In some embodiments in which the analyte comprises a carbohydrate or polysaccharide, the query probe comprises a carbohydrate-binding protein such as a lectin or a carbohydrate-binding antibody. In some embodiments, the presence of a specific glycosidic linkage or set of glycosidic linkages between carbohydrate monomers yields a distinguishable pattern of query probe binding. In some embodiments, the capture probe is a rabbit monoclonal antibody; and in some embodiments, the query probe is a mouse monoclonal antibody.

In some system embodiments, the analyte is stably immobilized to the surface by a surface-bound capture probe that stably binds the analyte. In some embodiments, the capture probe is a high-affinity antibody, an antibody fragment, or a nanobody. In some embodiments, the analyte is stably immobilized to the surface by a covalent bond cross-linking the analyte to the surface and/or to a surface-bound capture probe. In some embodiments, the analyte is subjected to thermal denaturation in the presence of a carrier prior to surface immobilization. In some embodiments, the analyte is subjected to chemical denaturation in the presence of a carrier prior to surface immobilization, e.g., the analyte is denatured with a denaturant such as urea, formamide, guanidinium chloride, high ionic strength, low ionic strength, high pH, low pH, or sodium dodecyl sulfate (SDS).

In some embodiments, the technology provides a system for detecting a protein analyte. For example, in some embodiments, the system comprises a capture probe that stably binds the protein analyte; and a query probe that transiently binds to the protein analyte. In some embodiments, the capture probe comprises an antibody. In some embodiments, the query probe comprises an antibody. In some embodiments, the query probe comprises an antigen-binding antibody fragment, monovalent Fab, nanobody, single-chain variable fragment antibody, an aptamer, or a low-affinity antibody. In some embodiments, the query probe comprises a label. In some embodiments, the query probe comprises a fluorescent label. In some embodiments, the capture probe is immobilized to a substrate. In some embodiments, the substrate is a substantially planar surface. In some embodiments, the substrate is a diffusible particle. In some embodiments, the system further comprises a detection component to detect transient binding of the query probe to the protein analyte. In some embodiments, the system further comprises a computer configured to receive and analyze kinetic data describing the association of the query probe with the protein analyte and dissociation of the query probe from the protein analyte.

In some embodiments, the system comprises a nanoparticle comprising a capture probe that stably binds the analyte; and a query probe that transiently binds to the analyte. In some embodiments, the system further comprises a collection component configured to collect the nanoparticles at a surface. In some embodiments, the nanoparticle has a diameter of 5 to 200 nanometers (e.g., 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 nm). In some embodiments, the nanoparticle is magnetic, paramagnetic, polar, charged, or has a density different than a medium comprising the nanoparticle. In some embodiments, the collection component produces a magnetic force, an electrical force, or an inertial force on the nanoparticle.

In some embodiments, the system comprises a capture probe that stably binds the analyte; a first query probe comprising a first label and that transiently binds the analyte; and a second query probe comprising a second label and that transiently binds the analyte. In some embodiments, the first query probe and the second query probe comprise the same probe moiety that transiently binds the analyte. In some embodiments, the first query probe and the second query probe comprise different probe moieties that transiently bind the analyte. In some embodiments, the first query probe and the second query probe comprise a Forster resonance energy transfer pair. In some embodiments, systems further comprise a detection component configured to detect colocalized transient binding of the first query probe and the second query probe with the analyte. In some embodiments, systems further comprise detection component configured to detect transient Førster resonance energy transfer between the first label and the second label.

In some embodiments, systems comprise a composition comprising a capture probe that stably binds the analyte; and a query probe that transiently binds to the analyte; and a temperature-control component configured to maintain the composition at 30-50° C. In some embodiments, the temperature is 30° C., 33° C., or 37° C. In some embodiments, systems further comprise a detection component configured to detect transient binding of the query probe to the analyte.

In some embodiments, systems comprise a composition comprising a capture probe that stably binds the analyte; a query probe that transiently binds to the analyte; and more than 100 mM ion concentration. In some embodiments, the ion is a monovalent cation. In some embodiments, the ion is a sodium ion. In some embodiments, the ion concentration is at least 500 mM.

In some embodiments, systems comprise a capture probe that stably binds the analyte; a query probe that transiently binds to the analyte; and a microfluidic device. In some embodiments, systems further comprise a detection component configured to detect transient binding of the query probe to the analyte.

In some embodiments, the system comprises an analyte covalently linked to a surface; and a query probe that transiently binds to the analyte. In some embodiments, the analyte is covalently linked to a capture probe and said capture probe is covalently linked to said surface. In some embodiments, the analyte is cross-linked to said capture probe by a product of a reaction with a N-hydroxysuccinimide ester, imidoester, haloacetyl, maleimide, or carbodiimide, or a derivative thereof. In some embodiments, the analyte is cross-linked to the capture probe by a product of a reaction produced by UV irradiation.

In some embodiments, the technology relates to a method for detecting an analyte using a system as described herein. For example, in some embodiments, methods comprise providing a system as described herein; and detecting the presence of and/or quantifying an analyte. In some embodiments, the analyte is biomarker for a disease. In some embodiments, the analyte is a biomarker for a cancer.

In some embodiments, the technology relates to a method for detecting and/or quantifying an analyte in a sample. For example, in some embodiments, methods comprise obtaining a sample from a subject; providing a system as described herein; and detecting and/or quantifying an analyte in said sample, wherein said analyte is a biomarker for a disease. In some embodiments, methods comprise providing a system as described herein; and detecting and/or quantifying an analyte in said sample, wherein said analyte is a biomarker for a cancer. In some embodiments, the sample is a biofluid. In some embodiments, the sample comprises and/or is prepared from blood, urine, mucus, saliva, semen, or tissue. In some embodiments, detecting and/or quantifying an analyte in the sample indicates that the subject has said disease. In some embodiments, the analyte comprises a protein, nucleic acid, or metabolite. In some embodiments, methods further comprise providing a result describing the presence and/or quantity of said analyte in said sample. In some embodiments, methods further comprise providing a positive control and/or a negative control. In some embodiments, methods further comprise providing a standard curve.

In some embodiments, the technology relates to use of a system as described herein to detect and/or quantify an analyte in a sample.

As discussed herein, some embodiments of the technology relate to microfluidic devices (e.g., methods and systems comprising and/or comprising use of a microfluidic device to detect an analyte). In particular, some embodiments relate to a system for detecting an analyte. For example, in some embodiments, systems comprise a capture probe that stably binds the analyte; a query probe that transiently binds to the analyte; and a microfluidic device comprising a substrate and a capture area in which the capture probe is immobilized. In some embodiments related to microfluidic devices, the capture probe comprises an antibody. In some embodiments related to microfluidic devices, the query probe comprises an antibody. In some embodiments related to microfluidic devices, the query probe comprises an antigen-binding antibody fragment, monovalent Fab, nanobody, single-chain variable fragment antibody, an aptamer, or a low-affinity antibody. In some embodiments related to microfluidic devices, the query probe comprises a label. In some embodiments related to microfluidic devices, the query probe comprises a fluorescent label.

In some embodiments related to microfluidic devices, the substrate of the microfluidic device is a substantially planar surface.

In some embodiments related to microfluidic devices, systems further comprise a detection component to detect transient binding of the query probe to the analyte.

In some embodiments related to microfluidic devices, systems further comprise a computer configured to receive and analyze kinetic data describing the association of the query probe with the protein analyte and dissociation of the query probe from the protein analyte.

In some embodiments related to microfluidic devices, the analyte is mixed in the capture area of the microfluidic device. In some embodiments related to microfluidic devices, the analyte is mixed by active and/or passive mixing systems (e.g., microstirrers, acoustic waves, microbubbles, periodic fluid pulsation, thermal mixing, electrokinetic mixing, ridges in the microfluidic device channel and/or capture area, herringbone structures in the microfluidic device channel and/or capture area and combinations thereof).

In some embodiments related to microfluidic devices, the analyte is immobilized to the substrate surface by a covalent bond cross-linking the analyte to the surface and/or to a surface-bound capture probe. In some embodiments related to microfluidic devices, the analyte is covalently linked to a capture probe and said capture probe is covalently linked to said surface. In some embodiments related to microfluidic devices, the analyte is cross-linked to said capture probe by a product of a reaction with a N-hydroxysuccinimide ester, imidoester, haloacetyl, maleimide, or carbodiimide, or a derivative thereof. In some embodiments related to microfluidic devices, the analyte is cross-linked to said capture probe by a product of a reaction produced by UV irradiation.

In some embodiments related to microfluidic devices, the system comprises two or more query probes that transiently bind to the analyte, each query probe comprising a different detectable label that distinguishes the binding of each query probe to the analyte. In some embodiments related to microfluidic devices, the first query probe and the second query probe comprise different probe moieties that transiently bind the analyte. In some embodiments related to microfluidic devices, the first query probe and the second query probe comprise a Førster resonance energy transfer pair.

In some embodiments related to microfluidic devices, systems further comprise a detection component configured to detect colocalized transient binding of the first query probe and the second query probe with the analyte. In some embodiments related to microfluidic devices, systems further comprise a detection component configured to detect transient Forster resonance energy transfer between the first label and the second label.

In some embodiments related to microfluidic devices, systems further comprise a temperature-control component configured to maintain the microfluidic device at approximately 25 to approximately 50° C.

In some embodiments related to microfluidic devices, the analyte is introduced into said microfluidic device in a solution containing an ion concentration of approximately 100 mM to approximately 1000 mM. In some embodiments related to microfluidic devices, the ion is a monovalent cation. In some embodiments related to microfluidic devices, the ion is a sodium ion. In some embodiments related to microfluidic devices, the ion concentration is at least 500 mM.

In some embodiments related to microfluidic devices, systems further comprise one or more component that concentrates the analyte, e.g., a component that provides electrophoretic stacking, electrophoretic focusing, flow confinement, cyclical reloading of analyte sample, and/or temperature gradient focusing of said analyte.

In some embodiments related to microfluidic devices, the analyte is a protein.

In some embodiments related to microfluidic devices, the technology provided herein relates to use of a system comprising a microfluidic device as described herein to detect and/or quantify an analyte in a sample.

In related embodiments, the technology provides methods comprising providing a system comprising a microfluidic device as described herein; and detecting and/or quantifying an analyte in said sample. In some embodiments related to microfluidic devices, methods further comprise an optional washing step after sample introduction. In some embodiments related to microfluidic devices, the sample is a biofluid, e.g., a sample comprising and/or that is prepared from blood, urine, mucus, saliva, semen, or tissue. In some embodiments related to microfluidic devices, detecting and/or quantifying an analyte in said sample indicates that the subject has said disease. In some embodiments related to microfluidic devices, the analyte comprises a protein, nucleic acid, or metabolite. In some embodiments related to microfluidic devices, methods further comprise providing a result describing the presence and/or quantity of said analyte in said sample. In some embodiments related to microfluidic devices, methods further comprise providing a positive control and/or a negative control. In some embodiments related to microfluidic devices, methods further comprise providing a standard curve.

As discussed herein, some embodiments of the technology relate to nanoparticles (e.g., methods and systems comprising and/or comprising use of a nanoparticle to detect an analyte). In particular, some embodiments relate to a system for detecting an analyte. For example, in some embodiments relating to nanoparticles, systems comprise a nanoparticle to which a capture probe that stably binds the analyte is attached; a query probe that transiently binds to the analyte; and a capture area.

In some embodiments related to nanoparticles, systems further comprise a collection component configured to collect the nanoparticles at the capture area.

In some embodiments related to nanoparticles, the nanoparticle has a diameter of 5 to 200 nanometers. In some embodiments related to nanoparticles, the nanoparticle is magnetic, paramagnetic, polar, charged, or has a density different than a medium comprising the nanoparticle.

In some embodiments related to nanoparticles, the collection component produces a magnetic force, an electrical force, or an inertial force on the nanoparticle.

In some embodiments related to nanoparticles, the capture probe comprises an antibody. In some embodiments related to nanoparticles, the query probe comprises an antibody. In some embodiments related to nanoparticles, the query probe comprises an antigen-binding antibody fragment, monovalent Fab, nanobody, single-chain variable fragment antibody, an aptamer, or a low-affinity antibody. In some embodiments, the query probe comprises a label. In some embodiments related to nanoparticles, the query probe comprises a fluorescent label.

In some embodiments related to nanoparticles, systems further comprise a detection component to detect transient binding of the query probe to the analyte. In some embodiments related to nanoparticles, systems further comprise a computer configured to receive and analyze kinetic data describing the association of the query probe with the protein analyte and dissociation of the query probe from the protein analyte.

In some embodiments related to nanoparticles, the analyte is immobilized to the substrate surface by a covalent bond cross-linking the analyte to the surface and/or to a surface-bound capture probe. In some embodiments related to nanoparticles, the analyte is covalently linked to a capture probe and said capture probe is covalently linked to said surface. In some embodiments related to nanoparticles, the analyte is cross-linked to said capture probe by a product of a reaction with a N-hydroxysuccinimide ester, imidoester, haloacetyl, maleimide, or carbodiimide, or a derivative thereof. In some embodiments related to nanoparticles, the analyte is cross-linked to said capture probe by a product of a reaction produced by UV irradiation. In some embodiments related to nanoparticles, the system comprises two or more query probes that transiently bind to the analyte, each query probe comprising a different detectable label that distinguishes the binding of each query probe to the analyte. In some embodiments related to nanoparticles, the first query probe and the second query probe comprise different probe moieties that transiently bind the analyte. In some embodiments related to nanoparticles, the first query probe and the second query probe comprise a Førster resonance energy transfer pair.

In some embodiments related to nanoparticles, systems further comprise a detection component configured to detect colocalized transient binding of the first query probe and the second query probe with the analyte. In some embodiments related to nanoparticles, systems further comprise a detection component configured to detect transient Førster resonance energy transfer between the first label and the second label. In some embodiments related to nanoparticles, systems further comprise a temperature-control component configured to maintain the microfluidic device at approximately 25 to approximately 50° C.

In some embodiments related to nanoparticles, the analyte is introduced into said microfluidic device in a solution containing an ion concentration of approximately 100 mM to approximately 1000 mM. In some embodiments related to nanoparticles, the ion is a monovalent cation. In some embodiments related to nanoparticles, the ion is a sodium ion. In some embodiments related to nanoparticles, the ion concentration is at least 500 mM.

In some embodiments related to nanoparticles, systems further comprise one or more component that concentrates the analyte, e.g., to provide electrophoretic stacking, electrophoretic focusing, flow confinement, cyclical reloading of analyte sample, and/or temperature gradient focusing of said analyte.

In some embodiments related to nanoparticles, the analyte is a protein.

In some embodiments, the technology relates to use of a system comprising a nanoparticle as described herein to detect and/or quantify an analyte in a sample.

In related embodiments, the technology provides methods of using a system comprising a nanoparticle as described herein. For example, in some embodiments, methods comprise providing a system comprising a nanoparticle as described herein; and detecting and/or quantifying an analyte in said sample. In some embodiments related to nanoparticles, methods further comprise an optional wash step after sample introduction. In some embodiments related to nanoparticles, the sample is a biofluid. In some embodiments related to nanoparticles, the sample comprises and/or is prepared from blood, urine, mucus, saliva, semen, or tissue. In some embodiments related to nanoparticles, detecting and/or quantifying an analyte in said sample indicates that the subject has said disease. In some embodiments related to nanoparticles, the analyte comprises a protein, nucleic acid, or metabolite. In some embodiments related to nanoparticles, methods further comprise providing a result describing the presence and/or quantity of said analyte in said sample. In some embodiments related to nanoparticles, methods further comprise providing a positive control and/or a negative control. In some embodiments related to nanoparticles, methods further comprise providing a standard curve.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings. The patent or application file contains at least one drawing executed in color. Copies of this patent or patent application publication with color drawings will be provided by the Office upon request and payment of the necessary fee.

FIG. 1 is a schematic drawing of the SiMREPS technology. An analyte is immobilized at a surface (e.g., through the use of a capture probe) and allowed to interact with a detectably binding query probe that is present in the adjacent solution. In the presence of captured analyte, each copy of analyte yields a characteristic pattern of query probe binding and dissociation that constitutes a kinetic fingerprint (top). In the absence of analyte, nonspecific binding of the query probe to the surface or to surface-immobilized molecules other than the analyte may occur, but these exhibit patterns of query probe binding and dissociation that are distinguishable from the kinetic fingerprint of the analyte, e.g., by having a different average number of binding and dissociation cycles per unit time or by having a different average (or median, or maximum, or minimum) dwell time in the bound or unbound states. The ability to distinguish kinetic fingerprints of specific binding from the nonspecific patterns increases as the number of observed binding events increases because the average properties of the kinetic fingerprints are increasingly well determined with more observed binding events per analyte molecule.

FIG. 2A is a schematic drawing showing detection of a protein analyte (target antigen) by SiMREPS. The repeated binding of a query probe (kinetic fingerprinting probe) to surface-captured antigen yields patterns of repeated binding that exhibit distinct kinetics from nonspecific interaction of probes with the surface or other matrix contaminants. The repeated binding of fluorescently labeled query probes can be visualized, for example, by total internal reflection fluorescence (TIRF) microscopy.

FIG. 2B (left) shows a single movie frame of a representative portion of a microscope field of view showing bright puncta at the locations where single fluorescent probes are bound at or near the imaging surface in a SiMREPS protein detection assay as described in FIG. 2A. FIG. 2B (right) shows plots of time-dependent patterns for the puncta indicated in the movie frame. The time-dependent patterns comprise periods of high and low fluorescence that indicate the binding and dissociation (or photobleaching) of query probes in the same location. Intensity-versus-time trajectories showing repeated binding and dissociation with statistical properties within a certain target range are determined to arise from interaction with the target antigen, resulting in detection of the target antigen.

FIG. 3A is a plot of representative intensity-versus-time trajectory data showing evidence of a detection probe repeatedly interacting with a single copy of the surface-immobilized target antigen VEGF-A.

FIG. 3B shows two scatter plots of N_{b+d} (number of binding and dissociation events observed per trajectory), τ_{on,median} (median lifetime in the query probe-bound state) and τ_{off,median} (median lifetime in the query probe-unbound state) for all intensity-versus-time trajectories observed within a single field of view in the presence of target antigen VEGF-A.

Dashed lines indicate thresholds (minimum or maximum) for accepting a trajectory as evidence of a single VEGF-A molecule. Points indicated by ‘+’ represent trajectories that do not pass filtering for intensity, signal-to-noise, and kinetics, and are not considered sufficient evidence to detect VEGF-A. Points indicated by circles represent trajectories that pass filtering and are accepted as evidence of the presence of individual VEGF-A molecules.

FIG. 3C shows two scatter plots of N_{b+d}, T_{on,median}, and T_{off,median} in the absence of VEGF-A. No trajectories pass filtering, indicating the absence of surface-bound target antigen.

FIG. 4A, FIG. 4B, and FIG. 4C show representative intensity-versus-time trajectories (top), N_{b+d}-versus-T_{on,median} plots (bottom left), and histograms of τ_{bound} (apparent lifetime of each query probe binding event to the target antigen) (bottom right) for detection of interleukin-6 (IL-6) by the same query probe at 22° C. (FIG. 4A), at 33° C. (FIG. 4B), and at 37° C. (FIG. 4C). The average bound-state lifetime (<τ_{bound}>) decreases by more than 10-fold with increasing temperature (e.g., from 27 seconds at 22° C. to 6 seconds at 33° C. to 2.3 seconds at 37° C.), providing for the observation of more binding and dissociation events in the same amount of time (or, equivalently, the same number of binding events in a shorter period of time).

FIG. 5A, FIG. 5B, and FIG. 5C show that increasing sodium ion concentration in the imaging buffer suppresses background binding and accelerates dissociation from the target antigen for a query probe used to detect plasminogen activation inhibitor-1 (PAI-1) by SiMREPS kinetic fingerprinting. FIG. 5A shows results of SiMREPS assay in low-salt PBS comprising 20 mM sodium ion for blank (left) and for a test composition comprising PAI-1 target antigen (right). FIG. 5B shows results of SiMREPS assay in PBS comprising 137 mM sodium ion for blank (left) and for a test composition comprising PAI-1 target antigen (right). FIG. 5C shows results of SiMREPS assay in PBS+500 mM NaCl comprising approximately 637 mM sodium ion for blank (left) and for a test composition comprising PAI-1 target antigen (right). N_{b+d}-versus-τ_{on,median} plots show that, as sodium ion concentration is increased from 20 mM (FIG. 5A) to 137 mM (FIG. 5B) to approximately 637 mM (FIG. 5C), the N_{b+d} values in the blank measurement become smaller on average, indicating less background binding of the query probe (FIG. 5A (left), FIG. 5B (left), and FIG. 5C (left)). Simultaneously, as sodium ion concentration is increased, the median bound-state lifetime of the query probe (τ_{on,median}) decreases, and the average N_{b+d} values observed in the presence of the target antigen PAI-1 increase. The combination of lower nonspecific binding and faster dissociation from the antigen results in kinetics of specific and nonspecific binding that are more easily distinguished at higher salt concentrations.

FIG. 6 shows a series of standard curves indicating quantitative detection of four antigens using SiMREPS kinetic fingerprinting with fluorescently labeled query probes. The matrix is animal serum (horse serum for PAI-1 and IL-6; chicken serum for VEGF-A and IL-34). Apparent limits of detection are 770 aM for PAI-1, 770 aM for IL-6, 3.6 fM for VEGF-A, and 6.5 fM for IL-34, which were calculated as three standard deviations above the mean of the blank. Error bars indicate one standard deviation of three measurements. Since the entire capture surface within each sample well comprises an area equivalent to approximately 1000 fields of view (FOV), the slopes of the standard curves indicate that between 250 and 1300 molecules are captured on the imaging surface per femtomolar of antigen in the 100-microliter samples, corresponding to a capture efficiency of 0.4-2.2%.

FIG. 7A is a schematic showing a wash-free protocol. The protocol was used for SiMREPS and provided quantitative detection of IL-6 in serum. In this protocol, the serum sample containing IL-6 was combined with the imaging solution comprising the query probe and then added to a coverslip that was pre-coated with a capture antibody. After a suitable incubation period (e.g. 30 minutes) the sample is imaged by TIRF microscopy to quantify IL-6.

FIG. 7B is a plot of data from kinetic fingerprinting of IL-6 with the Wash-Free protocol to provide a standard curve.

FIG. 7C is a correlation plot of IL-6 measurements in 34 patient-derived (human) serum samples by SiMREPS (no-wash protocol, 100-fold dilution for all samples) and ELISA (variable dilution factors, 4- or 64-fold, depending on analyte concentration). The correlation coefficient between the two methods is 0.999, despite the fact that the SiMREPS protocol avoids washing steps following sample introduction and uses up to 25-fold more dilute samples.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

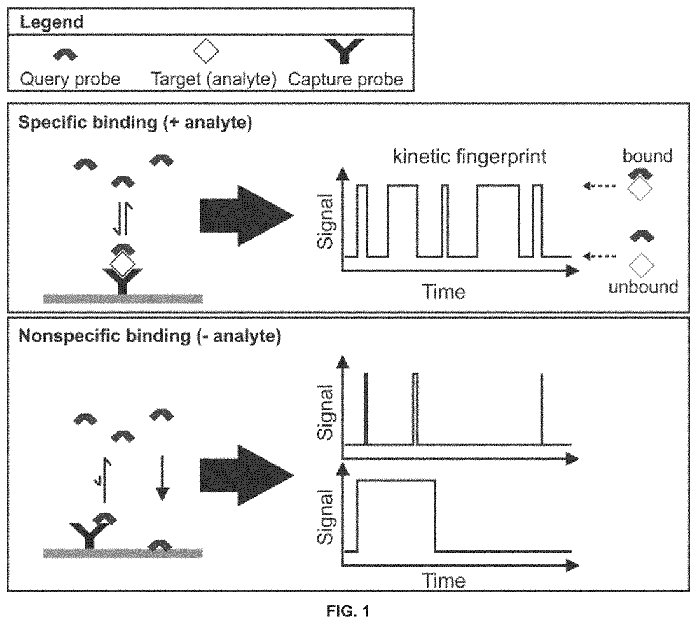

In some embodiments, the technology provided herein relates to detecting biomolecular analytes with transient (e.g., kinetic), rather than stable (equilibrium, thermodynamic), interactions with one or more query probes. The analytes are immobilized on a surface with a capture probe, then detected with the transiently binding query probe. In contrast to prior technologies, the technology described herein distinguishes between closely related analytes (e.g., phosphorylated and non-phosphorylated protein targets) with arbitrary precision by analyzing the kinetic behavior of the probe-target interaction. See FIG. 1.

In various embodiments, the assay conditions are controlled such that the interactions of the query probe with the analyte are made transient. For example, in some embodiments the technology comprises one or more of the following to provide conditions in which a transient interaction of probe and analyte occurs: (1) engineering a query probe such that it interacts weakly with the target (e.g., in the nanomolar affinity range); (2) controlling the temperature such that the query probe interacts weakly with the analyte; (3) controlling the solution conditions, e.g., ionic strength, ionic composition, addition of chaotropic agents, addition of competing probes, etc., such that the query probe interacts weakly with the analyte.

In some embodiments, the technology comprises use of, e.g., photonic forces and/or ultrasound energy. For example, in some embodiments photonic forces promote the concentration of material, especially larger particles, in a particular location. In some embodiments, ultrasound promotes mixing, e.g., to modulate the kinetics association, e.g., by increasing mixing rate beyond simple diffusion.

In some embodiments, binding of the query probe to the analyte is measured by total internal reflection fluorescence microscopy or another technique capable of single-molecule sensitivity.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the terms “subject” and “patient” refer to any organisms including plants, microorganisms, and animals (e.g., mammals such as dogs, cats, livestock, and humans).

As used herein, the term “sample” is used in its broadest sense. In some embodiments, a sample is or comprises an animal cell or tissue. In some embodiments, a sample includes a specimen or a culture (e.g., a microbiological culture) obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present technology.

As used herein, a “biological sample” refers to a sample of biological tissue or fluid. For instance, a biological sample may be a sample obtained from an animal (including a human); a fluid, solid, or tissue sample; as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs, rodents, etc. Examples of biological samples include sections of tissues, blood, blood fractions, plasma, serum, urine, or samples from other peripheral sources or cell cultures, cell colonies, single cells, or a collection of single cells. Furthermore, a biological sample includes pools or mixtures of the above mentioned samples. A biological sample may be provided by removing a sample of cells from a subject, but can also be provided by using a previously isolated sample. For example, a tissue sample can be removed from a subject suspected of having a disease by conventional biopsy techniques. In some embodiments, a blood sample is taken from a subject. A biological sample from a patient means a sample from a subject suspected to be affected by a disease.

Environmental samples include environmental material such as surface matter, soil, water, and industrial samples, as well as samples obtained from food and dairy processing instruments, apparatus, equipment, utensils, disposable and non-disposable items. These examples are not to be construed as limiting the sample types applicable to the present invention.

As used herein, the term “label” refers to any atom, molecule, molecular complex (e.g., metal chelate), or colloidal particle (e.g., quantum dot, nanoparticle, microparticle, etc.) that can be used to provide a detectable (preferably quantifiable) effect, and that can be attached to a nucleic acid or protein. Labels include, but are not limited to, dyes (e.g., optically-detectable labels, fluorescent dyes or moieties, etc.); radiolabels such as ³²P; binding moieties such as biotin; haptens such as digoxgenin; luminogenic, phosphorescent, optically-detectable, or fluorogenic moieties; mass tags; and fluorescent dyes alone or in combination with moieties that can suppress or shift emission spectra by Førster resonance energy transfer (FRET), which is also known as fluorescence resonance energy transfer. See, e.g., Jones and Bradshaw (2019) “Resonance Energy Transfer: From Fundamental Theory to Recent Applications” Frontiers in Physics Volume 7, article 100, incorporated herein by reference. Labels may provide signals detectable by fluorescence, luminescence, radioactivity, colorimetry, gravimetry, X-ray diffraction or absorption, magnetism, enzymatic activity, characteristics of mass or behavior affected by mass (e.g., MALDI time-of-flight mass spectrometry; fluorescence polarization), and the like. A label may be a charged moiety (positive or negative charge) or, alternatively, may be charge neutral. Labels can include or consist of nucleic acid or protein sequence, so long as the sequence comprising the label is detectable.

As used herein the term “fluorophore” will be understood to refer to both fluorophores and luminophores and chemical agents that quench fluorescent or luminescent emissions. Further, as used herein, a “fluorophore” refers to any species possessing a fluorescent property when appropriately stimulated. The stimulation that elicits fluorescence is typically illumination; however, other types of stimulation (e.g., collisional) are also considered herein. The terms “fluorophore”, “fluor”, “fluorescent moiety”, “fluorescent dye”, and “fluorescent group” are used interchangeably. In some embodiments, a fluorescent label comprises a fluorophore as described below in the section entitled “Fluorescent labels”.

As used herein, the term “support” or “solid support” refers to a matrix on or in which nucleic acid molecules, microparticles, and the like may be immobilized, e.g., to which they may be covalently or noncovalently attached or in or on which they may be partially or completely embedded so that they are largely or entirely prevented from diffusing freely or moving with respect to one another.

As used herein, the term “moiety” refers to one of two or more parts into which something may be divided, such as, for example, the various parts of an oligonucleotide, a molecule, a chemical group, a domain, a probe, etc.

As used herein, the term “nucleic acid” or a “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogeneous or homogeneous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino, locked nucleic acid (LNA), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand.

As used herein, the term “nucleotide analog” refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner and herein incorporated by reference); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872; each of which is herein incorporated by reference); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include nucleotides having modification on the sugar moiety, such as dideoxy nucleotides and 2′-O-methyl nucleotides. Nucleotide analogs include modified forms of deoxyribonucleotides as well as ribonucleotides.

As used herein, the term “peptide nucleic acid” means a DNA mimic that incorporates a peptide-like polyamide backbone.

As used herein, the terms “complementary” or “complementarity” are used in reference to polynucleotides (e.g., a sequence of nucleotides such as an oligonucleotide capture probe, query probe or an analyte that is a nucleic acid) related by the base-pairing rules. For example, for the sequence “5′-A-G-T-3′” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

In some contexts, the term “complementarity” and related terms (e.g., “complementary”, “complement”) refers to the nucleotides of a nucleic acid sequence that can bind to another nucleic acid sequence through hydrogen bonds, e.g., nucleotides that are capable of base pairing, e.g., by Watson-Crick base pairing or other base pairing. Nucleotides that can form base pairs, e.g., that are complementary to one another, are the pairs: cytosine and guanine, thymine and adenine, adenine and uracil, and guanine and uracil. The percentage complementarity need not be calculated over the entire length of a nucleic acid sequence. The percentage of complementarity may be limited to a specific region of which the nucleic acid sequences that are base-paired, e.g., starting from a first base-paired nucleotide and ending at a last base-paired nucleotide. The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

Thus, in some embodiments, “complementary” refers to a first nucleobase sequence that is at least 60%, 65%, 70%, 75%, 80%, 85%, 90%, 95%, 97%, 98%, or 99% identical to the complement of a second nucleobase sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases, or that the two sequences hybridize under stringent hybridization conditions. “Fully complementary” means each nucleobase of a first nucleic acid is capable of pairing with each nucleobase at a corresponding position in a second nucleic acid. For example, in certain embodiments, an oligonucleotide wherein each nucleobase has complementarity to a nucleic acid has a nucleobase sequence that is identical to the complement of the nucleic acid over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, or more nucleobases.

As used herein, the term “mismatch” refers to a nucleobase of a first nucleic acid that is not capable of pairing with a nucleobase at a corresponding position of a second nucleic acid.

As used herein, the term “domain” when used in reference to a polypeptide refers to a subsection of the polypeptide which possesses a unique structural and/or functional characteristic; typically, this characteristic is similar across diverse polypeptides. The subsection typically comprises contiguous amino acids, although it may also comprise amino acids which act in concert or which are in close proximity due to folding or other configurations. Examples of a protein domain include transmembrane domains, glycosylation sites, etc.

As used herein, the term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

As used herein, the term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product. Thus, the terms “variant” and “mutant” when used in reference to a nucleotide sequence refer to an nucleic acid sequence that differs by one or more nucleotides from another, usually related nucleotide acid sequence. A “variation” is a difference between two different nucleotide sequences; in some embodiments, one sequence is a reference sequence.

As used herein, the term “allele” refers to different variations in a gene; the variations include but are not limited to variants and mutants, polymorphic loci and single nucleotide polymorphic loci, frameshift and splice mutations. An allele may occur naturally in a population, or it might arise during the lifetime of any particular individual of the population.

As used herein, the term “hybridization” is used in reference to the pairing of complementary nucleic acids. Hybridization and the strength of hybridization (e.g., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, e.g., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modern biology.

As used herein, the term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41*(% G+C), when a nucleic acid is in aqueous solution at 1 M NaCl (see, e.g., Anderson and Young, Quantitative Filter Hybridization, in Nucleic Acid Hybridization (1985). Other references (e.g., Allawi and SantaLucia, Biochemistry 36: 10581-94 (1997), incorporated herein by reference) include more sophisticated computations which account for structural, environmental, and sequence characteristics to calculate Tₘ. For example, in some embodiments these computations provide an improved estimate of Tₘ for short nucleic acid probes and targets (e.g., as used in the examples).

As used herein, the terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein. Where the term “amino acid sequence” is recited herein to refer to an amino acid sequence of a protein molecule, “amino acid sequence” and like terms such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein. Conventional one and three-letter amino acid codes are used herein as follows—Alanine: Ala, A; Arginine: Arg, R; Asparagine: Asn, N; Aspartate: Asp, D; Cysteine: Cys, C; Glutamate: Glu, E; Glutamine: Gln, Q; Glycine: Gly, G; Histidine: His, H; Isoleucine: Ile, I; Leucine: Leu, L; Lysine: Lys, K; Methionine: Met, M; Phenylalanine: Phe, F; Proline: Pro, P; Serine: Ser, S; Threonine: Thr, T; Tryptophan Trp, W; Tyrosine: Tyr, Y; Valine Val, V. As used herein, the codes Xaa and X refer to any amino acid.

As used herein, the terms “variant” and “mutant” when used in reference to a polypeptide refer to an amino acid sequence that differs by one or more amino acids from another, usually related, polypeptide.

As used herein, the term “melting” when used in reference to a nucleic acid refers to the dissociation of a double-stranded nucleic acid or region of a nucleic acid into a single-stranded nucleic acid or region of a nucleic acid.

As used herein, a “query probe” or “reader probe” is any entity (e.g., molecule, biomolecule, etc.) that recognizes an analyte (e.g., binds to an analyte, e.g., binds specifically to an analyte). In exemplary embodiments, the query probe is a protein (e.g., an antibody) that recognizes an analyte. In some other exemplary embodiments, the query probe is a nucleic acid that recognizes an analyte (e.g., a DNA, an RNA, a nucleic acid comprising DNA and RNA, a nucleic acid comprising modified bases and/or modified linkages between bases; e.g., a nucleic acid as described hereinabove, a nucleic acid aptamer). In some embodiments, the query probe is labeled, e.g., with a detectable label such as, e.g., a fluorescent moiety as described herein. In some embodiments, the query probe comprises more than one type of molecule (e.g., more than one of a protein, a nucleic acid, a chemical linker or a chemical moiety).

As used herein, an “event” refers to an instance of a query probe binding to an analyte or an instance of query probe dissociation from an analyte, e.g., as measured by monitoring a detectable property indicating the binding of a query probe to an analyte and/or the dissociation of a query probe from an analyte.

As used herein, a “capture probe” is any entity (e.g., molecule, biomolecule, etc.) that recognizes an analyte (e.g., binds to an analyte, e.g., binds specifically to an analyte) and links the analyte to a solid support. In some embodiments, the capture probe is a protein (e.g., an antibody) that recognizes an analyte. In some embodiments, a capture probe is a nucleic acid that recognizes an analyte (e.g., a DNA, an RNA, a nucleic acid comprising DNA and RNA, a nucleic acid comprising modified bases and/or modified linkages between bases; e.g., a nucleic acid as described hereinabove, a nucleic acid aptamer). In some embodiments, a capture probe is labeled, e.g., with a detectable label such as, e.g., a fluorescent moiety as described herein. In some embodiments, the capture probe comprises more than one type of molecule (e.g., more than one of a protein, a nucleic acid, a chemical linker or a chemical moiety).

As used herein, the term “sensitivity” refers to the probability that an assay gives a positive result for the analyte when the sample comprises the analyte. Sensitivity is calculated as the number of true positive results divided by the sum of the true positives and false negatives. Sensitivity is a measure of how well an assay detects an analyte.

As used herein, the term “specificity” refers to the probability that an assay gives a negative result when the sample does not comprise the analyte. Specificity is calculated as the number of true negative results divided by the sum of the true negatives and false positives. Specificity is a measure of how well a method of the present invention excludes samples that do not comprise an analyte from those that do comprise the analyte.

As used herein, the “equilibrium constant” (K_{eq}), the “equilibrium association constant” (Kₐ), and “association binding constant” (or “binding constant” (K_{B})) are used interchangeably for the following binding reaction of A and B at equilibrium:

A+B [symbol] AB

where A and B are two entities that associate with each other (e.g., capture probe and analyte, query probe and analyte) and K_{eq}=[AB]/([A]×[B]). The dissociation constant K_{D}=1/K_{B}. The K_{D} is a useful way to describe the affinity of a one binding partner A for a partner B with which it associates, e.g., the number K_{D} represents the concentration of A or B that is required to yield a significant amount of AB. K_{eq}=k_{off}/kₒₙ; K_{D}=k_{off}/kₒₙ. Accordingly, the dissociation constant, K_{D}, and the association constant, K_{A}, are quantitative measures of affinity. At equilibrium, A and B are in equilibrium with A-B complex, and the rate constants, kₐ and k_{d}, quantify the rates of the individual forward and backward reactions of the equilibrium state:

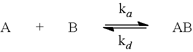

At equilibrium, kₐ [A][B]=k_{d} [AB]. The dissociation constant, K_{D}, is given by K_{D}=k_{d}/k_{d}=[A][B]/[AB]. K_{D} has units of concentration, e.g., M, mM, μM, nM, pM, etc. When comparing affinities expressed as K_{D}, a greater affinity is indicated by a lower value. The association constant, K_{A}, is given by K_{A}=1/K_{D}=[AB]/[A][B]. K_{A} has units of inverse concentration, most typically M⁻¹, mM⁻¹, μM⁻¹, nM⁻¹, pM⁻¹, etc.

As used herein, a “significant amount” of the product of two entities that associate with each other, e.g., formation of AB from A and B according to the equation above, refers to a concentration of AB that is equal to or greater than the free concentration of A or B, whichever is smaller.

As used herein, “nanomolar affinity range” refers to the association of two components that has an equilibrium dissociation constant K_{D} (e.g., ratio of k_{off}/kₒₙ) in the nanomolar range, e.g., a dissociation constant (K_{D}) of 1×10⁻¹⁰ to 1×10⁻⁵M (e.g., in some embodiments 1×10⁻⁹ to 1×10⁻⁶ M). The dissociation constant has molar units (M). The smaller the dissociation constant, the higher the affinity between two components (e.g., capture probe and analyte; query probe and analyte).

As used herein, a “weak affinity” or “weak binding” or “weak association” refers to an association having a K_{D} of approximately 100 nanomolar (e.g., approximately 10, 20, 30, 40, 50, 60, 70, 80, 90, 100, 110, 120, 130, 140, 150, 160, 170, 180, 190, 200, 300, 400, or 500 nanomolar) and/or, in some embodiments, in the range of 1 nanomolar to 10 micromolar.

The terms “specific binding” or “specifically binding” when used in reference to the interaction of two components A and B that associate with one another refers to an association of A and B having a K_{D} that is smaller than the K_{D} for the interaction of A or B with other similar components in the solution, e.g., at least one other molecular species in the solution that is not A or B.

As used herein, the word “presence” or “absence” (or, alternatively, “present” or “absent”) is used in a relative sense to describe the amount or level of a particular entity (e.g., an analyte). For example, when an analyte is said to be “present” in a sample, it means the level or amount of this analyte is above a pre-determined threshold; conversely, when an analyte is said to be “absent” in a sample, it means the level or amount of this analyte is below a pre-determined threshold. The pre-determined threshold may be the threshold for detectability associated with the particular test used to detect the analyte or any other threshold. When an analyte is “detected” in a sample it is “present” in the sample; when an analyte is “not detected” it is “absent” from the sample. Further, a sample in which an analyte is “detected” or in which the analyte is “present” is a sample that is “positive” for the analyte. A sample in which an analyte is “not detected” or in which the analyte is “absent” is a sample that is “negative” for the analyte.

As used herein, an “increase” or a “decrease” refers to a detectable (e.g., measured) positive or negative change in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

The term “detection assay” refers to an assay for detecting the presence or absence of an analyte or the activity or effect of an analyte or for detecting the presence or absence of a variant of an analyte.

As used herein, the term “system” denotes a set of components, real or abstract, comprising a whole where each component interacts with or is related to at least one other component within the whole.

In some embodiments the technology comprises an antibody component or moiety, e.g., a capture probe and/or a query probe comprising an antibody or fragments or derivatives thereof. As used herein, an “antibody”, also known as an “immunoglobulin” (e.g., IgG, IgM, IgA, IgD, IgE), comprises two heavy chains linked to each other by disulfide bonds and two light chains, each of which is linked to a heavy chain by a disulfide bond. The specificity of an antibody resides in the structural complementarity between the antigen combining site of the antibody (or paratope) and the antigen determinant (or epitope). Antigen combining sites are made up of residues that are primarily from the hypervariable or complementarity determining regions (CDRs). Occasionally, residues from nonhypervariable or framework regions influence the overall domain structure and hence the combining site. Some embodiments comprise a fragment of an antibody, e.g., any protein or polypeptide-containing molecule that comprises at least a portion of an immunoglobulin molecule such as to permit specific interaction between said molecule and an antigen. The portion of an immunoglobulin molecule may include, but is not limited to, at least one complementarity determining region (CDR) of a heavy or light chain or a ligand binding portion thereof, a heavy chain or light chain variable region, a heavy chain or light chain constant region, a framework region, or any portion thereof. Such fragments may be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. The various portions of antibodies can be joined together chemically by conventional techniques or can be prepared as a contiguous protein using genetic engineering techniques.

Fragments of antibodies include, but are not limited to, Fab (e.g., by papain digestion), F(ab′)₂ (e.g., by pepsin digestion), Fab′ (e.g., by pepsin digestion and partial reduction) and Fv or scFv (e.g., by molecular biology techniques) fragments.

A Fab fragment can be obtained by treating an antibody with the protease papain. Also, the Fab may be produced by inserting DNA encoding a Fab of the antibody into a vector for prokaryotic expression system or for eukaryotic expression system and introducing the vector into a prokaryote or eukaryote to express the Fab. A F(ab′)₂ may be obtained by treating an antibody with the protease pepsin. Also, the F(ab′)₂ can be produced by binding a Fab′ via a thioether bond or a disulfide bond. A Fab may be obtained by treating F(ab′)₂ with a reducing agent, e.g., dithiothreitol. Also, a Fab′ can be produced by inserting DNA encoding a Fab′ fragment of the antibody into an expression vector for a prokaryote or an expression vector for a eukaryote and introducing the vector into a prokaryote or eukaryote for its expression. A Fv fragment may be produced by restricted cleavage by pepsin, e.g., at 4° C. and pH 4.0. (a method called “cold pepsin digestion”). The Fv fragment consists of the heavy chain variable domain (V_{H}) and the light chain variable domain (V_{L}) held together by strong noncovalent interaction. A scFv fragment may be produced by obtaining cDNA encoding the V_{H} and V_{L} domains as previously described, constructing DNA encoding scFv, inserting the DNA into an expression vector for prokaryote or an expression vector for eukaryote, and then introducing the expression vector into a prokaryote or eukaryote to express the scFv.

In general, antibodies can usually be raised to any antigen, using the many conventional techniques now well known in the art.

As used herein, the term “conjugated” refers to when one molecule or agent is physically or chemically coupled or adhered to another molecule or agent. Examples of conjugation include covalent linkage and electrostatic complexation. The terms “complexed”, “complexed with”, and “conjugated” are used interchangeably herein.

As used herein, a “stable interaction” or referring to a “stably bound” interaction refers to an association that is relatively persistent under the thermodynamic equilibrium conditions of the interaction. In some embodiments, a “stable interaction” is an interaction between two components having a K_{D} that is smaller than approximately 10⁻⁹ M or, in some embodiments a K_{D} that is smaller than 10⁻⁸ M. In some embodiments, a “stable interaction” has a dissociation rate constant k_{off} that is smaller than 1 per hour or, in some embodiments, a dissociation rate constant k_{off} that is smaller than 1 per minute. In some embodiments, a “stable interaction” is defined as not being a “transient interaction” and a “transient interaction” is defined as not being a “stable interaction”. In some embodiments, a “stable interaction” includes interactions mediated by covalent bonds and other interactions that are not typically described by a K_{D} value but that involve an average association lifetime between two entities that is longer than approximately 1 minute (e.g., 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 seconds) per each interaction.

In some embodiments, the distinction between a “stable interaction” and a “transient interaction” is determined by a cutoff value of K_{D} and/or k_{off} and/or another kinetic or thermodynamic value describing the associations, wherein the cutoff is used to discriminate between stable and transient interactions that might otherwise be characterized differently if described in absolute terms of a K_{D} and/or k_{off} or another kinetic or thermodynamic value describing the associations. For example, a “stable interaction” characterized by a K_{D} value might also be characterized as a “transient interaction” in the context of another interaction that is even more stable. One of skill in the art would understand other relative comparisons of stable and transient interactions, e.g., that a “transient interaction” characterized by a K_{D} value might also be characterized as a “stable interaction” in the context of another interaction that is even more transient (less stable).

As used herein, the terms “stable interaction”, “stable binding”, and “stable association” are used interchangeably. As used herein, the terms “transient interaction”, “transient binding”, and “transient association” are used interchangeably.

As used herein, the term “affinity” refers to the strength of interaction (e.g., binding) of one entity (e.g., molecule) with another entity (e.g., molecule), e.g., an antibody with an antigen. In some embodiments, affinity depends on the closeness of stereochemical fit between entities, on the size of the area of contact between them, on the distribution of charged and hydrophobic groups, etc.

As used herein, the term “irreversible interaction” refers to an interaction (e.g., association, binding, etc.) having a dissociation half-life longer than the incubation time, e.g., in some embodiments, a time that is 1 to 10 minutes (e.g., 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 seconds, or longer).

As used herein, “moiety” refers to one of two or more parts into which something may be divided, such as, for example, the various parts of an oligonucleotide, a molecule, a chemical group, a domain, a probe, an “R” group, a polypeptide, etc.

As used herein, in some embodiments a “signal” is a time-varying quantity associated with one or more properties of a sample that is assayed, e.g., the binding of a query probe to an analyte and/or dissociation of a query probe from an analyte. A signal can be continuous in the time domain or discrete in the time domain. As a mathematical abstraction, the domain of a continuous-time signal is the set of real numbers (or an interval thereof) and the domain of a discrete-time signal is the set of integers (or an interval thereof). Discrete signals often arise via “digital sampling” of continuous signals. For example, an audio signal consists of a continually fluctuating voltage on a line that can be digitized by reading the voltage level on the line at a regular interval, e.g., every 50 microseconds. The resulting stream of numbers is stored as a discrete-time digital signal. In some embodiments, the signal is recorded as a function of location in space (e.g., x, y coordinates; e.g., x, y, z coordinates). In some embodiments, the signal is recorded as a function of time. In some embodiments, the signal is recorded as a function of time and location.

As used herein, the term “algorithm” is a broad term and is used in its ordinary sense, including, but not limited to, the computational processes (for example, programs) involved in transforming information from one state to another, for example using computer processing.

### Description

Although the disclosure herein refers to certain illustrated embodiments, it is to be understood that these embodiments are presented by way of example and not by way of limitation.

### SiMREPS

As used herein, the term “single-molecule recognition through equilibrium Poisson sampling” and its abbreviation “SiMREPS” refers to an amplification-free, single-molecule detection approach for identifying and counting analytes in biofluids by “kinetic fingerprinting”. As used herein, the term “kinetic fingerprinting” is used interchangeably with the term “SiMREPS”. The technology is described in U.S. Pat. No. 10,093,967; U.S. patent application Ser. Nos. 16/154,045; 16/076,853; 15/914,729; 16/219,070; and Int'l Pat. App. No. PCT/US19/43022, each of which is incorporated herein by reference. See also Johnson-Buck et al. (2015) “Kinetic fingerprinting to identify and count single nucleic acids” Nature Biotechnology 33: 730-32, incorporated herein by reference.

In brief, the SiMREPS technology comprises directly observing the repeated binding of fluorescent probes to surface-captured analytes (e.g., nucleic acid, protein, etc.), which produces a specific (e.g., for nucleic acid, a sequence-specific) kinetic fingerprint. The kinetic fingerprint identifies the analyte with high-confidence at single-molecule resolution. The kinetic fingerprint overcomes previous technologies limited by thermodynamic specificity barriers and thereby minimizes and/or eliminates false positives. Thus, the SiMREPS technology provides an ultra-high specificity that finds use in detecting, e.g., rare analytes such as rare or low-abundance mutant DNA alleles. Prior work has shown that SiMREPS is capable of single-nucleotide discrimination (see, e.g., Johnson-Buck et al. (2015) “Kinetic fingerprinting to identify and count single nucleic acids” Nat. Biotechnol. 33: 730-32; Su et al. (2017) “Single-Molecule Counting of Point Mutations by Transient DNA Binding” Sci Rep 7: 43824, each of which is incorporated herein by reference). See FIG. 1.

The technology provides for the detection of analytes, e.g., in the presence of similar analytes and, in some embodiments, background noise. In some embodiments, signal originating from the transient binding of the query probe to the analyte is distinguishable from the signal produced by unbound query probe (e.g., by observing, monitoring, and/or recording a localized change in signal intensity during the binding event). In some embodiments, observing the transient binding of the query probe (e.g., a fluorescently labeled query probe) to the analyte is provided by a technology such as, e.g., total internal reflection fluorescence (TIRF) or near-TIRF microscopy, zero-mode waveguides (ZMWs), light sheet microscopy, stimulated emission depletion (STED) microscopy, or confocal microscopy. In some embodiments, the technology provided herein uses query probes having a fluorescence emission that is quenched when not bound to the analyte and/or a fluorescence emission that is dequenched when bound to the analyte.

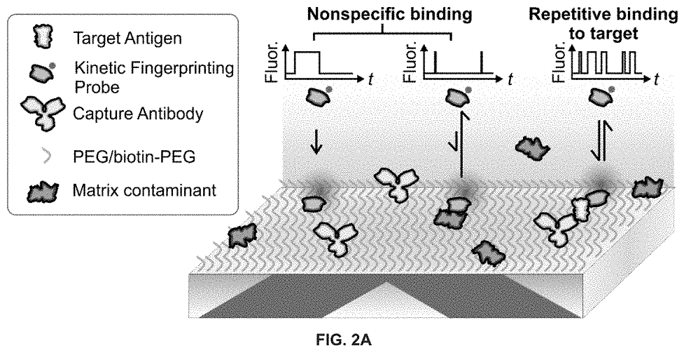

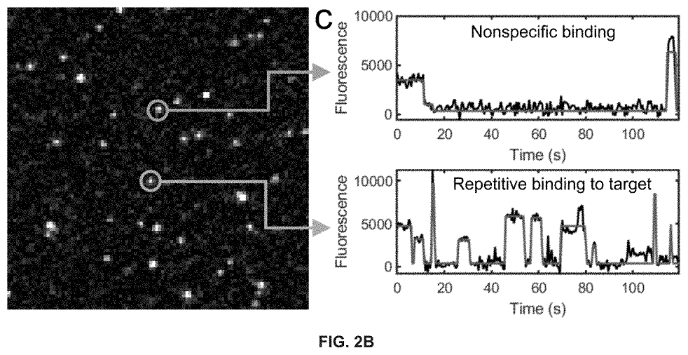

In particular embodiments, the technology finds use in detecting a protein analyte (e.g., an antigen) using a capture probe and/or a query probe comprising an antibody or antigen-binding antibody fragment (e.g., IgG, (Fab)₂, monovalent Fab, nanobody, or single-chain variable fragment antibody), an aptamer (e.g., a nucleic acid or peptide aptamer), or a naturally occurring binding partner of the protein analyte, a peptide sequence of a protein analyte, or a post-translational modification of the protein analyte. See FIG. 2A and FIG. 2B.

The technology comprises locating and/or observing the transient binding of a query probe to an analyte within a discrete region of an area and/or a discreet region of a volume that is observed, e.g., at particular spatial coordinates in a plane or a volume. In some embodiments, the error in determining the spatial coordinates of a binding or dissociation event (e.g., due to limited signal, detector noise, or spatial binning in the detector) is small (e.g., minimized, eliminated) relative to the average spacing between immobilized (e.g., surface-bound) analytes. In some embodiments comprising use of wide-field fluorescence microscopy, measurement errors are minimized and/or eliminated by use of effective detector pixel dimensions in the specimen plane that are not larger than the average distance between immobilized (e.g., surface-bound) analytes and that many fluorescent photons (in some embodiments, more than 100, e.g., more than 80, 85, 90, 95, 100, 105, 110, 115, 120, 125, or 130 or more) are collected per time point of detection.

In some embodiments, the detectable (e.g., fluorescent) query probe produces a fluorescence emission signal when it is close to the surface of the solid support (e.g., within about 100 nm of the surface of the solid support). When unbound, query probes quickly diffuse and thus are not individually detected; accordingly, when in the unbound state, the query probes produce a low level of diffuse background fluorescence. Consequently, in some embodiments detection of bound query probes comprises use of total internal reflection fluorescence microscopy (TIRF), HiLo microscopy (see, e.g., U.S. Pat. App. Pub. No. 20090084980, European Patent No. 2300983 B1, Int'l Pat. App. Pub. No. WO2014018584 A1, and Int'l Pat. App. Pub. No. WO2014018584 A1, each of which is incorporated herein by reference), confocal scanning microscopy, or other technologies comprising illumination schemes that illuminate (e.g., excite) only those query probe molecules near or on the surface of the solid support. Thus, in some embodiments, only query probes that are bound to an immobilized target near or on the surface produce a point-like emission signal (e.g., a “spot”) that can be confirmed as originating from a single molecule.

In some embodiments, the query probe comprises a fluorescent label having an emission wavelength. Detection of fluorescence emission at the emission wavelength of the fluorescent label indicates that the query probe is bound to an immobilized analyte. Binding of the query probe to the analyte is a “binding event”. In some embodiments of the technology, a binding event has a fluorescence emission having a measured intensity greater than a defined threshold. For example, in some embodiments a binding event has a fluorescence intensity that is above the background fluorescence intensity (e.g., the fluorescence intensity observed in the absence of an analyte). In some embodiments, a binding event has a fluorescence intensity that is at least 1, 2, 3, 4 or more standard deviations above the background fluorescence intensity (e.g., the fluorescence intensity observed in the absence of an analyte). In some embodiments, a binding event has a fluorescence intensity that is at least 2 standard deviations above the background fluorescence intensity (e.g., the fluorescence intensity observed in the absence of an analyte). In some embodiments, a binding event has a fluorescence intensity that is at least 1.5, 2, 3, 4, or 5 times the background fluorescence intensity (e.g., the mean fluorescence intensity observed in the absence of an analyte).

Accordingly, in some embodiments detecting fluorescence at the emission wavelength of the query probe that has an intensity above the defined threshold (e.g., at least 2 standard deviations greater than background intensity) indicates that a binding event has occurred (e.g., at a discrete location on the solid support where an analyte is immobilized). Also, in some embodiments detecting fluorescence at the emission wavelength of the query probe that has an intensity above the defined threshold (e.g., at least 2 standard deviations greater than background intensity) indicates that a binding event has started. Accordingly, in some embodiments detecting an absence of fluorescence at the emission wavelength of the query probe that has an intensity above the defined threshold (e.g., at least 2 standard deviations greater than background intensity) indicates that a binding event has ended (e.g., the query probe has dissociated from the analyte). The length of time between when the binding event started and when the binding event ended (e.g., the length of time that fluorescence at the emission wavelength of the fluorescent probe having an intensity above the defined threshold (e.g., at least 2 standard deviations greater than background intensity) is detected) is the dwell time of the binding event. A “transition” refers to the binding and dissociation of a query probe to the analyte (e.g., an on/off event), e.g., a query probe dissociating from a bound state or a query probe associating with an analyte from the unbound state.

Methods according to the technology comprise counting the number of query probe binding events that occur at each discrete location (e.g., at a position identified by x, y coordinates) on the solid support during a defined time interval that is the “acquisition time” (e.g., a time interval that is tens to hundreds to thousands of seconds, e.g., 5, 10, 15, 20, 25, 30, 35, 40, 45, 50, 55, or 60 seconds; e.g., 5, 10, 15, 20, 25, 30, 35, 40, 45, 50, 55, or 0 minutes; e.g., 1, 1.5, 2, 2.5, or 3 hours). In some embodiments, the acquisition time is approximately 1 to 10 seconds to 1 to 10 minutes (e.g., approximately 1 to 100 seconds, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 seconds, e.g., 1 to 100 minutes, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 minutes).

Further, the length of time the query probe remains bound to the analyte during a binding event is the “dwell time” of the binding event. The number of binding events detected during the acquisition time and/or the lengths of the dwell times recorded for the binding events is/are characteristic of a query probe binding to an analyte and thus provide an indication that the analyte is immobilized at said discrete location and thus that the analyte is present in the sample.

Binding of the query probe to the immobilized analyte and/or and dissociation of the query probe from the immobilized analyte is/are monitored (e.g., using a light source to excite the fluorescent probe and detecting fluorescence emission from a bound query probe, e.g., using a fluorescence microscope) and/or recorded during a defined time interval (e.g., during the acquisition time). The number of times the query probe binds to the analyte during the acquisition time and/or the length of time the query probe remains bound to the analyte during each binding event and the length of time the query probe remains unbound to the analyte between each binding event (e.g., the “dwell times” in the bound and unbound states, respectively) are determined, e.g., by the use of a computer and software (e.g., to analyze the data using a hidden Markov model and Poisson statistics).

In some embodiments, positive and/or negative control samples are measured (e.g., a control sample known to comprise or not to comprise a target). Fluorescence detected in a negative control sample is “background fluorescence” or “background (fluorescence) intensity” or “baseline”.

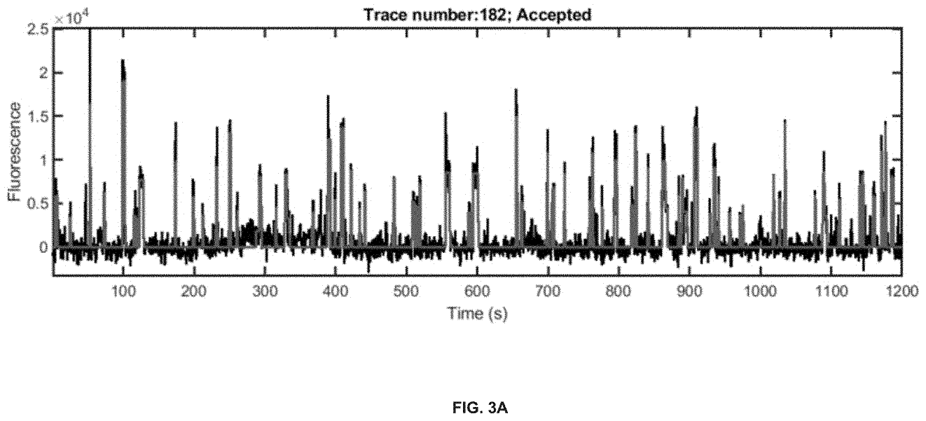

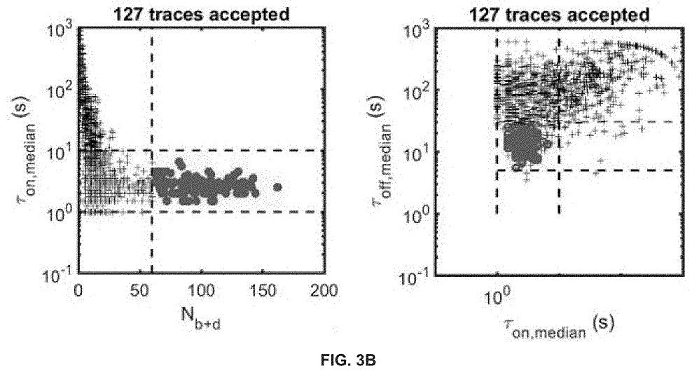

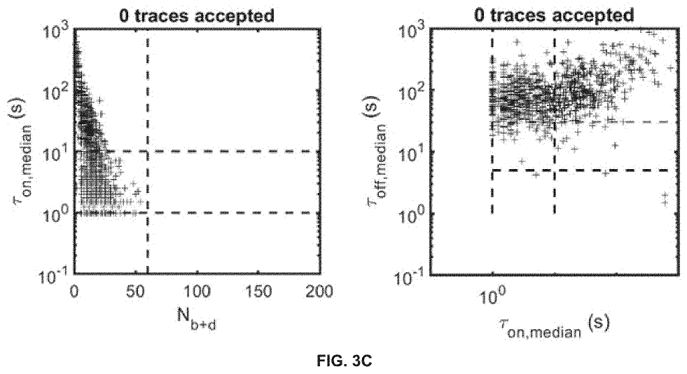

In some embodiments, data comprising measurements of fluorescence intensity at the emission wavelength of the query probe are recorded as a function of time. See FIG. 3A. In some embodiments, the number of binding events and the dwell times of binding events (e.g. for each immobilized analyte) are determined from the data (e.g., by determining the number of times and the lengths of time the fluorescence intensity is above a threshold background fluorescence intensity). In some embodiments, transitions (e.g., binding and dissociation of one or more query probes) are counted for each discrete location on the solid support where an analyte is immobilized. In some embodiments, a threshold number of transitions is used to discriminate the presence of an analyte at a discrete location on the solid support from background signal, non-analyte, and/or spurious binding of the query probe. See FIG. 3B and FIG. 3C.

In some embodiments, a distribution of the number of transitions for each immobilized target is determined—e.g., the number of transitions is counted for each immobilized analyte observed. In some embodiments a histogram is produced. In some embodiments, characteristic parameters of the distribution are determined, e.g., the mean, median, peak, shape, etc. of the distribution are determined. In some embodiments, data and/or parameters (e.g., fluorescence data (e.g., fluorescence data in the time domain), kinetic data, characteristic parameters of the distribution, etc.) are analyzed by algorithms that recognize patterns and regularities in data, e.g., using artificial intelligence, pattern recognition, machine learning, statistical inference, neural nets, etc. In some embodiments, the analysis comprises use of a frequentist analysis and in some embodiments the analysis comprises use of a Bayesian analysis. In some embodiments, pattern recognition systems are trained using known “training” data (e.g., using supervised learning) and in some embodiments algorithms are used to discover previously unknown patterns (e.g., unsupervised learning). See, e.g., Duda, et al. (2001) Pattern classification (2nd edition), Wiley, New York; Bishop (2006) Pattern Recognition and Machine Learning, Springer.

Pattern recognition (e.g., using training sets, supervised learning, unsupervised learning, and analysis of unknown samples) associates identified patterns with analytes such that particular patterns provide a “fingerprint” of particular analytes that find use in detection, quantification, and identification of analytes.

In some embodiments, the distribution produced from an analyte is significantly different than a distribution produced from a non-analyte or the distribution produced in the absence of an analyte. In some embodiments, a mean number of transitions is determined for the plurality of immobilized analytes. In some embodiments, the mean number of transitions observed for a sample comprising an analyte is approximately linearly related as a function of time and has a positive slope (e.g., the mean number of transitions increases approximately linearly as a function of time).

In some embodiments, the data are treated using statistics (e.g., Poisson statistics) to determine the probability of a transition occurring as a function of time at each discrete location on the solid support. In some particular embodiments, a relatively constant probability of a transition event occurring as a function of time at a discrete location on the solid support indicates the presence of an analyte at said discrete location on the solid support. In some embodiments, a correlation coefficient relating event number and elapsed time is calculated from the probability of a transition event occurring as a function of time at a discrete location on the solid support. In some embodiments, a correlation coefficient relating event number and elapsed time greater than 0.95 when calculated from the probability of a transition event occurring as a function of time at a discrete location on the solid support indicates the presence of an analyte at said discrete location on the solid support.

In some embodiments, dwell times of bound query probe (τₒₙ) and unbound query probe (τ_{off}) are used to identify the presence of an analyte in a sample and/or to distinguish a sample comprising an analyte from a sample comprising a non-analyte and/or not comprising the analyte. For example, the τₒₙ for an analyte is greater than the τₒₙ for a non-analyte; and, the τ_{off} for an analyte is smaller than the τ_{off} for a non-analyte. In some embodiments, measuring τₒₙ and τ_{off} for a negative control and for a sample indicates the presence or absence of the analyte in the sample. In some embodiments, a plurality of τₒₙ and τ_{off} values is determined for each of a plurality of spots imaged on a solid support, e.g., for a control (e.g., positive and/or negative control) and a sample suspected of comprising an analyte. In some embodiments, a mean τₒₙ and/or τ_{off} is determined for each of a plurality of spots imaged on a solid support, e.g., for a control (e.g., positive and/or negative control) and a sample suspected of comprising an analyte. In some embodiments, a plot of τₒₙ versus τ_{off} (e.g., mean τₒₙ and τ_{off}, time-averaged τₒₙ and τ_{off}, etc.) for all imaged spots indicates the presence or absence of the analyte in the sample. See FIG. 3B and FIG. 3C.

As described herein, the technology detects analytes by a kinetic detection technology. Accordingly, particular embodiments of the technology are related to detecting an analyte by analyzing the kinetics of the interaction of a query probe with the analyte to be detected. For the interaction of a query probe Q (e.g., at an equilibrium concentration [Q]) with an analyte T (e.g., at an equilibrium concentration [T]), the kinetic rate constant kₒₙ describes the time-dependent formation of the complex QT comprising the probe Q hybridized to the analyte T. In particular embodiments, while the formation of the QT complex is associated with a second order rate constant that is dependent on the concentration of query probe and has units of M⁻¹min⁻¹ (or the like), the formation of the QT complex is sufficiently described by a kₒₙ that is a pseudo-first order rate constant associated with the formation of the QT complex. Thus, as used herein, kₒₙ is an apparent (“pseudo”) first-order rate constant.

Likewise, the kinetic rate constant k_{off} describes the time-dependent dissociation of the complex QT into the probe Q and the analyte T. Kinetic rates are typically provided herein in units of min⁻¹ or s⁻¹. The “dwell time” of the query probe Q in the bound state (τₒₙ) is the time interval (e.g., length of time) that the probe Q is hybridized to the analyte T during each instance of query probe Q binding to the analyte T to form the QT complex. The “dwell time” of the query probe Q in the unbound state (τ_{off}) is the time interval (e.g., length of time) that the probe Q is not hybridized to the analyte T between each instance of query probe Q binding to the analyte to form the QT complex (e.g., the time the query probe Q is dissociated from the analyte T between successive binding events of the query probe Q to the analyte T). Dwell times may be provided as averages or weighted averages integrating over numerous binding and non-binding events.

Further, in some embodiments, the repeated, stochastic binding of probes (e.g., detectably labeled query probes (e.g., fluorescent probes) to analytes is modeled as a Poisson process occurring with constant probability per unit time and in which the standard deviation in the number of binding and dissociation events per unit time (N_{b+d}) increases as (N_{b+d})^{1/2}. Thus, the statistical noise becomes a smaller fraction of N_{b+d} as the observation time is increased. Accordingly, the observation is lengthened as needed in some embodiments to achieve discrimination between target and off-target binding. And, as the acquisition time is increased, the signal and background peaks in the N_{b+d} histogram become increasingly separated and the width of the signal distribution increases as the square root of N^{b+d}, consistent with kinetic Monte Carlo simulations.

Further, in some embodiments assay conditions are controlled to tune the kinetic behavior to improve discrimination of query probe binding events to the analyte from background binding. For example, in some embodiments the technology comprises control of assay conditions such as, e.g., using a query probe that is designed to interact weakly with the analyte (e.g., in the nanomolar affinity range); controlling the temperature such that the query probe interacts weakly with the analyte; controlling the solution conditions, e.g., ionic strength, ionic composition, presence of organic compounds, addition of chaotropic agents, and addition of competing probes.

Some embodiments provide a method of identifying an analyte by repetitive query probe binding. In some embodiments, methods comprise immobilizing an analyte to a solid support. In some embodiments, the solid support is a surface (e.g., a substantially planar surface, a rounded surface), e.g., a surface in contact with a bulk solution, e.g., a bulk solution comprising analyte. In some embodiments, the solid support is a freely diffusible solid support (e.g., a bead, a colloidal particle, e.g., a colloidal particle having a diameter of approximately 10-1000 nm (e.g., 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, or 1000 nm)), e.g., that freely diffuses within the bulk solution, e.g., a bulk solution comprising the analyte. In some embodiments, immobilizing an analyte to a solid support comprises covalent interaction between the solid support and analyte. In some embodiments, immobilizing an analyte to a solid support comprises non-covalent interaction between the solid support and analyte. In some embodiments, the analyte (e.g., a molecule, e.g., a molecule such as, e.g., a protein, peptide, nucleic acid, small molecule, lipid, metabolite, drug, etc.) is stably immobilized to a surface and methods comprise repetitive (e.g., transient, low-affinity) binding of a query probe to the analyte. In some embodiments, methods comprise detecting the repetitive (e.g., transient, low-affinity) binding of a query probe to the analyte. In some embodiments, methods comprise generating a dataset comprising a signal produced from query probe binding to the analyte (e.g., a dataset of query probe signal as a function of time) and information (e.g., coordinates, e.g., x, y coordinates) describing the spatial position on the surface of the query probe binding to the analyte. In some embodiments, the dataset is processed (e.g., manipulated, transformed, visualized, etc.), e.g., to improve the spatial resolution of the query probe binding events. For example, in some embodiments, the dataset (e.g., comprising query probe signal as a function of time and information (e.g., coordinates, e.g., x, y coordinates) describing the spatial position on the surface of the query probe binding to the analyte) is subjected to processing. In some embodiments, the processing comprises a frame-by-frame subtraction process to generate differential intensity profiles showing query probe binding or dissociation events within each frame of the time series data. Data collected during the development of the technology described herein indicate that the differential intensity profiles have a higher resolution than the query probe binding signal vs. position map. In some embodiments, after determining the spatial position (e.g., x, y coordinates) of each query probe binding and/or dissociation event, a plurality of events is clustered according to spatial position and the kinetics of the events within each cluster are subjected to statistical analysis to determine whether the cluster of events originates from a given analyte.

For instance, some embodiments of methods for quantifying one or more surface-immobilized or diffusing analytes comprise one or more steps including, e.g., measuring the signal of one or more transiently binding query probes to the immobilized analyte(s) with single-molecule sensitivity. In some embodiments, methods comprise tracking (e.g., detecting and/or recording the position of) analytes independently from query probe binding. In some embodiments, the methods further comprise calculating the time-dependent probe binding signal intensity changes at the surface as a function of position (e.g., x, y position). In some embodiments, calculating the time-dependent query probe binding signal intensity changes at the surface as a function of position (e.g., x, y position) produces a “differential intensity profile” for query probe binding to the analyte. In some embodiments, the methods comprise determining the position (e.g., x, y position) of each query probe binding and dissociation event (“event”) with sub-pixel accuracy from a differential intensity profile. In some embodiments, methods comprise grouping events into local clusters by position (e.g., x, y position) on the surface, e.g., to associate events for a single immobilized analyte. In some embodiments, the methods comprise calculating kinetic parameters from each local cluster of events to determine whether the cluster originates from a particular analyte, e.g., from transient probe binding to a particular analyte.

Embodiments of methods are not limited in the analyte that is detected. For example, in some embodiments the analyte is polypeptide, e.g., a protein or a peptide. In some embodiments, the analyte is a nucleic acid. In some embodiments, the analyte is a small molecule.

In some embodiments, the interaction between the analyte and the query probe is distinguishably influenced by a covalent modification of the analyte. For example, in some embodiments, the analyte is a polypeptide comprising a post-translational modification, e.g., a protein or a peptide comprising a post-translational modification. In some embodiments, a post-translational modification of a polypeptide affects the transient binding of a query probe with the analyte, e.g., the query probe signal is a function of the presence or absence of the post-translational modification on the polypeptide. For example, in some embodiments, the analyte is a nucleic acid comprising an epigenetic modification, e.g., a nucleic acid comprising a methylated base. In some embodiments, the analyte is a nucleic acid comprising a covalent modification to a nucleobase, a ribose, or a deoxyribose moiety of the analyte.

In some embodiments, a modification of a nucleic acid affects the transient binding of a query probe with the analyte, e.g., the query probe signal is a function of the presence or absence of the modification on the nucleic acid. In some embodiments, the transient interaction between the post-translational modification and the query probe is mediated by a chemical affinity tag, e.g., a chemical affinity tag comprising a nucleic acid.

In some embodiments, the query probe is a nucleic acid or an aptamer. In some embodiments, the query probe is a low-affinity antibody, antibody fragment, or nanobody. In some embodiments, the query probe is a DNA-binding protein, RNA-binding protein, or a DNA-binding ribonucleoprotein complex.

In some embodiments, the position, e.g., the (x,y) position, of each binding or dissociation event is determined by subjecting the differential intensity profile to centroid determination, least-squares fitting to a Gaussian function, least-square fitting to an airy disk function, least-squares fitting to a polynomial function (e.g., a parabola), or maximum likelihood estimation.

In some embodiments, the capture probe is a high-affinity antibody, antibody fragment, or nanobody. In some embodiments, the capture probe is a nucleic acid. In some embodiments, capture is mediated by a covalent bond cross-linking the analyte to the surface and/or to a surface-bound capture probe. In some embodiments, the analyte is subjected to thermal denaturation in the presence of a carrier prior to surface immobilization. In some embodiments, the analyte is subjected to chemical denaturation in the presence of a carrier prior to surface immobilization, e.g., the analyte is denatured with a denaturant such as urea, formamide, guanidinium chloride, high ionic strength, low ionic strength, high pH, low pH, or sodium dodecyl sulfate (SDS).

### Analytes

The technology is not limited in the analyte that is detected, quantified, identified, or otherwise characterized (e.g., presence, absence, amount, concentration, state). The term “analyte” as used herein is a broad term and is used in its ordinary sense, including, without limitation, to refer to a substance or chemical constituent in a sample such as a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte comprises a salt, sugars, protein, fat, vitamin, or hormone. In some embodiments, the analyte is naturally present in a biological sample (e.g., is “endogenous”); for example, in some embodiments, the analyte is a metabolic product, a hormone, an antigen, an antibody, and the like. Alternatively, in some embodiments, the analyte is introduced into a biological organism (e.g., is “exogenous), for example, a drug, drug metabolite, a drug precursor (e.g., prodrug), a contrast agent for imaging, a radioisotope, a chemical agent, etc. The metabolic products of drugs and pharmaceutical compositions are also contemplated analytes.

In some embodiments, the analyte is a polypeptide, a nucleic acid, a small molecule, a lipid, a carbohydrate, a polysaccharide, a fatty acid, a phospholipid, a glycolipid, a sphingolipid, an organic molecule, an inorganic molecule, cofactor, pharmaceutical, bioactive agent, a cell, a tissue, an organism, etc. In some embodiments, the analyte comprises a polypeptide, a nucleic acid, a small molecule, a lipid, a carbohydrate, a polysaccharide, a fatty acid, a phospholipid, a glycolipid, a sphingolipid, an organic molecule, an inorganic molecule, cofactor, pharmaceutical, bioactive agent, a cell, a tissue, an organism, etc. In some embodiments, the analyte comprises a combination of one or more of a polypeptide, a nucleic acid, a small molecule, a lipid, a carbohydrate, a polysaccharide, a fatty acid, a phospholipid, a glycolipid, a sphingolipid, an organic molecule, an inorganic molecule, cofactor, pharmaceutical, bioactive agent, a cell, a tissue, an organism, etc.

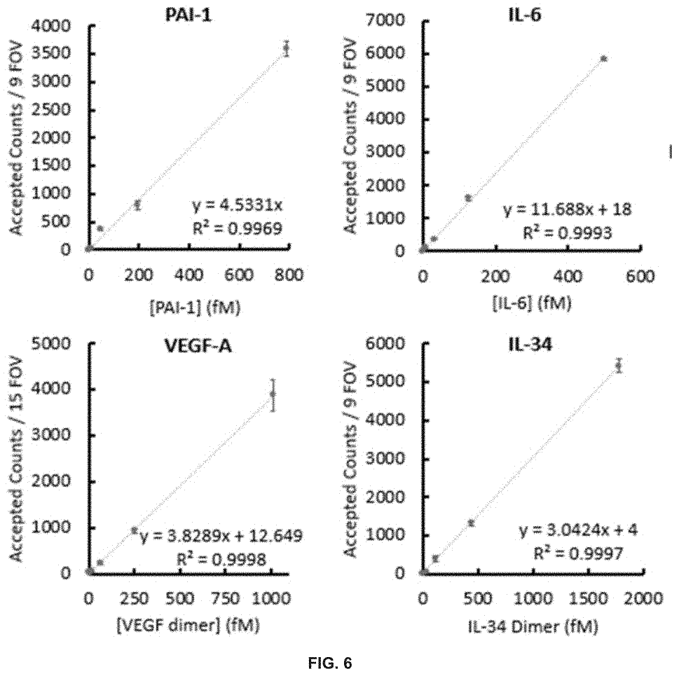

In some embodiments, the analyte is part of a multimolecular complex, e.g., a multiprotein complex, a nucleic acid/protein complex, a molecular machine, an organelle (e.g., a cell-free mitochondrion, e.g., in plasma; a plastid; golgi, endoplasmic reticulum, vacuole, peroxisome, lysosome, and/or nucleus), cell, virus particle, tissue, organism, or any macromolecular complex or structure or other entity that can be captured and is amenable to analysis by the technology described herein (e.g., a ribosome, spliceosome, vault, proteasome, DNA polymerase III holoenzyme, RNA polymerase II holoenzyme, symmetric viral capsids, GroEL/GroES; membrane protein complexes: photosystem I, ATP synthase, nucleosome, centriole and microtubule-organizing center (MTOC), cytoskeleton, flagellum, nucleolus, stress granule, germ cell granule, or neuronal transport granule). For example, in some embodiments a multimolecular complex is isolated and the technology finds use in characterizing, identifying, quantifying, and/or detecting one or more molecules (analytes) associated with (e.g., that is a component of) the multimolecular complex. In some embodiments an extracellular vesicle is isolated and the technology finds use in characterizing, identifying, quantifying, and/or detecting one or more molecules (analytes) associated with the vesicle. In some embodiments, the technology finds use in characterizing, identifying, quantifying, and/or detecting a protein (e.g., a surface protein) and/or an analytes present inside the vesicle, e.g., a protein, nucleic acid, or other analyte described herein. In some embodiments, the vesicle is fixed and permeabilized prior to analysis. In some embodiments, the protein is an antigen and/or comprises an antigen and the assay comprises use of a query probe comprising an antibody and/or a capture probe comprising an antibody. The technology finds use in detecting a wide variety of protein analytes (e.g., antigens). See FIG. 6.

In some embodiments, the analyte is chemically modified to provide a site for query probe binding. For instance, in some embodiments, beta-elimination of phosphoserine and phosphothreonine under strongly basic conditions is used to introduce an alkene, followed by Michael addition of a nucleophile such as a dithiol to the alkene. The remaining free thiol is then used for conjugation to a maleimide-containing oligonucleotide with a sequence complementary to an oligonucleotide query probe. The post-translational modifications phosphoserine and phosphothreonine may then be probed using the query probe and analyzed as described herein.

As used herein, the terms “detect an analyte” or “detect a substance” will be understood to encompass direct detection of the analyte itself or indirect detection of the analyte (e.g., by detecting a by-product).

### Capture

Embodiments of the technology comprise capture of an analyte. In some embodiments, the analyte is captured and immobilized. In some embodiments, the analyte is stably attached to a solid support. In some embodiments, the solid support is immobile relative to a bulk liquid phase contacting the solid support. In some embodiments, the solid support is diffusible within a bulk liquid phase contacting the solid support.

The technology is not limited in the capture probe. In some embodiments, the capture probe is an antibody (e.g., a monoclonal antibody) or antibody fragment. In some embodiments, the capture probe is an antibody or antibody fragment that has been engineered for increased affinity for the analyte. In some embodiments, the capture probe is a nanobody, a DNA-binding protein or protein domain, a methylation binding domain (MBD), a kinase, a phosphatase, an acetylase, a deacetylase, an enzyme, or a polypeptide. In some embodiments, the capture probe is an oligonucleotide that interacts with the analyte. In some embodiments, the capture probe is a pharmaceutical agent, e.g., a drug or other bioactive molecule. In some embodiments, the capture probe is a metal ion complex. In some embodiments, the capture probe is a methyl-binding domain (e.g., MBD1). In some embodiments, the capture probe is labeled with a detectable label as described herein. In some embodiments, the capture probe is covalently linked to the detectable label. In some embodiments, the capture probe is indirectly and/or non-covalently linked and/or associated with the detectable label. In some embodiments, the detectable label is fluorescent.

In some embodiments, the capture probe is an antibody (e.g., a monoclonal antibody) or antibody fragment. In some embodiments in which the analyte comprises a carbohydrate or polysaccharide, the capture probe comprises a carbohydrate-binding protein such as a lectin or a carbohydrate-binding antibody.

In some embodiments, stable attachment of the analyte to a surface or other solid substrate is provided by a high-affinity or irreversible interaction (e.g., as used herein, an “irreversible interaction” refers to an interaction having a dissociation half-life longer than the observation time, e.g., in some embodiments, a time that is 1 to 5 minutes (e.g., 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 seconds, or longer). The technology is not limited in the components and/or methods used for capture of the analyte. For example, the stable attachment is provided by a variety of methods, including but not limited to one or more of the following.

In some embodiments, an analyte is immobilized by a surface-bound capture probe with a dissociation constant (K_{D}) for the analyte smaller than approximately 1 nanomolar (nM) (e.g., less than 1.5, 1.4, 1.3, 1.2, 1.1, 1.0, 0.9, 0.8, 0.7, 0.6, 0.5 nanomolar) and a dissociation rate constant for the analyte that is smaller than approximately 1 min⁻¹ (e.g., less than approximately 1.5, 1.4, 1.3, 1.2, 1.1, 1.0, 0.9, 0.8, 0.7, 0.6, 0.5 min⁻¹). Exemplary surface-bound capture probes include, e.g., an antibody, antibody fragment, nanobody, or other protein; a high-affinity DNA-binding protein or ribonucleoprotein complex such as Cas9, dCas9, Cpf1, transcription factors, or transcription activator-like effector nucleases (TALENs); an oligonucleotide; a small organic molecule; or a metal ion complex.

In some embodiments, an analyte is immobilized by direct noncovalent attachment to a surface (e.g., by interactions between the analyte and the surface, e.g., a glass surface or a nylon, nitrocellulose, or polyvinylidene difluoride membrane).

In some embodiments, an analyte is immobilized by chemical linking (e.g., by a covalent bond) of the analyte to the solid support. In some embodiments, the analyte is chemically linked to the solid support by, e.g., a carbodiimide, a N-hydroxysuccinimide esters (NHS) ester, a maleimide, a haloacetyl group, a hydrazide, or an alkoxyamine. In some embodiments, an analyte is immobilized by radiation (e.g., ultraviolet light)-induced cross-linking of the analyte to the surface and/or to a capture probe attached to the surface. In some embodiments, the capture probe is a monoclonal antibody. In some embodiments in which the analyte comprises a carbohydrate or polysaccharide, the capture probe comprises a carbohydrate-binding protein such as a lectin or a carbohydrate-binding antibody.

In some embodiments, the technology comprises forming one or more covalent bonds to cross-link the analyte to a surface-immobilized capture probe, thus preventing dissociation of the analyte from the surface prior to or during the measurements. The technology is not limited in the chemistry used to produce a cross-link between an analyte and a capture probe. For example, embodiments incubating the analyte-capture probe complex with a reactive chemical such as an NHS ester derivative (e.g., disuccinimidyl tartrate, disuccinimidyl suberate, or disuccinimidyl glutarate), imidoester derivative (e.g., dimethyl pimelimidate, dimethyl suberimidate), haloacetyl derivative (e.g., succinimidyl iodoacetate), maleimide derivative (e.g., succinimidyl 4-(N-maleimidomethyl)cyclohexane-1-carboxylate), or carbodiimide derivative (e.g., 1-ethyl-3-(3-dimethylaminopropyl) carbodiimide); or by irradiation of the analyte-capture probe complex with UV light. After cross-liking the analyte to the surface-immobilized capture probe, the captured analyte is detected by SiMREPS,

Alternatively, instead of immobilizing the analyte to a solid support that is relatively stationary with respect to a bulk phase that contacts the solid support as described above, some embodiments provide that the analyte is associated with a freely diffusing particle that diffuses within the bulk fluid phase contacting the freely diffusing particle. Accordingly, in some embodiments, the analyte is covalently or noncovalently bound to a freely diffusing substrate. In some embodiments, the freely diffusing substrate is, e.g., a colloidal particle (e.g., a particle having a diameter of approximately 10-1000 nm (e.g., 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, or 1000 nm)). In some embodiments, the freely diffusing substrate comprises and/or is made of, e.g., polystyrene, silica, dextran, gold, or DNA origami. In some embodiments, the analyte is associated with a freely diffusing particle that diffuses slowly relative to the diffusion of the query probe, e.g., the analyte has a diffusion coefficient that is less than approximately 10% (e.g., less than 15, 14, 13, 12, 11, 10.5, 10.4, 10.3, 10.2, 10.1, 10.0, 9.9, 9.8, 9.7, 9.6, 9.5, or 9.0% or less) of the diffusion coefficient of the query probe.

Furthermore, in some embodiments the analyte is associated with a freely diffusing particle and the location of the analyte is observable and/or recordable independently of observing and/or recording query probe binding. For example, in some embodiments a detectable label (e.g., a fluorophore, fluorescent protein, quantum dot) is covalently or noncovalently attached to the analyte, e.g., for detection and localization of the analyte. Accordingly, in some embodiments the position of the analyte and the position of query probe binding events are simultaneously and independently measured.

In some embodiments, the analyte is associated with a surface by capturing the analyte using a nanoparticle comprising a capture probe and collecting (e.g., immobilizing) the particles comprising captured analyte at a surface for subsequent SiMREPS analysis. In some embodiments, the nanoparticle has a diameter of approximately 5 to approximately 200 nanometers and is collected (e.g., immobilized) at a surface by applying force to a composition comprising the nanoparticle (e.g., magnetic, inertial (e.g., centrifugal), electrical).

### Query

Embodiments of the technology comprise a query probe (e.g., a detectably labeled query probe) that binds transiently and repeatedly to the analyte, e.g., a query probe that binds to and dissociates from the analyte several (e.g., greater than 1, 2, 3, 4, 5, 6, 7, 8, 9, 10, or more) times per observation window. In some embodiments, the query probe has a dissociation constant (K_{D}) for the analyte of larger than approximately 1 nanomolar (e.g., greater than 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or more nanomolar) under the assay conditions. In some embodiments, the query probe has a binding and/or a dissociation constant for the analyte that is larger than approximately 1 min⁻¹ (e.g., greater than 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 or more min⁻¹).

The technology is not limited in the query probe. In some embodiments, the query probe is an antibody or antibody fragment. In some embodiments, the query probe is a low-affinity antibody or antibody fragment. In some embodiments, the query probe is an antibody that has been engineered to have a reduced affinity. In some embodiments, the query probe is a nanobody, a DNA-binding protein or protein domain, a methylation binding domain (MBD), a kinase, a phosphatase, an acetylase, a deacetylase, an enzyme, or a polypeptide. In some embodiments, the query probe is an oligonucleotide that interacts with the analyte. For example, in some embodiments the query probe is an oligonucleotide that hybridizes to the analyte to form a duplex that has a melting temperature that is within approximately 10 degrees Celsius of the temperature at which the observations are made (e.g., approximately 7-12 nucleotides for observation that is performed at room temperature). In some embodiments, the query probe is a mononucleotide. In some embodiments, the query probe is a small organic molecule (e.g., a molecule having a molecular weight that is less than approximately 2000 daltons, e.g., less than 2100, 2050, 2000, 1950, 1900, 1850, 1800, 1750, 1700, 1650, 1600, 1550, 1500 daltons, or less). In some embodiments, the query probe is a pharmaceutical agent, e.g., a drug or other bioactive molecule. In some embodiments, the query probe is a metal ion complex. In some embodiments, the query probe is a methyl-binding domain (e.g., MBD1). In some embodiments, the query probe is labeled with a detectable label as described herein. In some embodiments, the query probe is covalently linked to the detectable label. In some embodiments, the query probe is indirectly and/or non-covalently linked and/or associated with the detectable label. In some embodiments, the detectable label is fluorescent.

In some embodiments, the query probe is an antibody (e.g., a monoclonal antibody) or antibody fragment.

In some embodiments in which the analyte comprises a carbohydrate or polysaccharide, the query probe comprises a carbohydrate-binding protein such as a lectin or a carbohydrate-binding antibody.

In some embodiments, the technology relates to use of SiMREPS for detecting the presence, absence, and/or quantity of an analyte using query probes labeled with two or more different labels (e.g., fluorophores). In some embodiments, the technology comprises use of two or more query probes that are specific for the same analyte and that comprise two or more different labels.

In some embodiments, the two or more query probes comprise a first query probe comprising a first label and a second query probe comprising a second label (and, optionally, a third, fourth, fifth, sixth, seventh, eighth, ninth, tenth, etc. query probe comprising, respectively, a third, fourth, fifth, sixth, seventh, eighth, ninth, tenth, etc. label). In some embodiments, the first query probe is a different query probe than the second query probe (e.g., a composition comprises different query probes comprising different labels). In some embodiments, the first query probe is the same query probe as the second query probe (e.g., a first portion of the query probe molecules comprises a first label and a second portion of the query probe molecules comprises a second label). In some embodiments, the first label and the second label are a FRET pair. In some embodiments, the technology comprises detecting colocalized signals produced by the two or more labels and/or detecting FRET between two labels.

### Detection

The technology provides for the detection of analytes, e.g., in the presence of similar analytes and, in some embodiments, background noise. In some embodiments, signal originating from the transient binding of the query probe to the analyte is distinguishable from the signal produced by unbound query probe (e.g., by observing, monitoring, and/or recording a localized change in signal intensity during the binding event). In some embodiments, observing the transient binding of the query probe (e.g., a fluorescently labeled query probe) to the analyte is provided by a technology such as, e.g., total internal reflection fluorescence (TIRF) or near-TIRF microscopy, zero-mode waveguides (ZMWs), light sheet microscopy, stimulated emission depletion (STED) microscopy, or confocal microscopy. In some embodiments, the technology provided herein uses query probes having a fluorescence emission that is quenched when not bound to the analyte and/or a fluorescence emission that is dequenched when bound to the analyte.

The technology comprises locating and/or observing the transient binding of a query probe to an analyte within a discrete region of an area and/or a discreet region of a volume that is observed, e.g., at particular spatial coordinates in a plane or a volume. In some embodiments, the error in determining the spatial coordinates of a binding or dissociation event (e.g., due to limited signal, detector noise, or spatial binning in the detector) is small (e.g., minimized, eliminated) relative to the average spacing between immobilized (e.g., surface-bound) analytes. In some embodiments comprising use of wide-field fluorescence microscopy, measurement errors are minimized and/or eliminated by use of effective detector pixel dimensions in the specimen plane that are not larger than the average distance between immobilized (e.g., surface-bound) analytes and that many fluorescent photons (in some embodiments, more than 100, e.g., more than 80, 85, 90, 95, 100, 105, 110, 115, 120, 125, or 130 or more) are collected per time point of detection.

In some embodiments, the detectable (e.g., fluorescent) query probe produces a fluorescence emission signal when it is close to the surface of the solid support (e.g., within about 100 nm of the surface of the solid support). When unbound, query probes quickly diffuse and thus are not individually detected; accordingly, when in the unbound state, the query probes produce a low level of diffuse background fluorescence. Consequently, in some embodiments detection of bound query probes comprises use of total internal reflection fluorescence microscopy (TIRF), HiLo microscopy (see, e.g., U.S. Pat. App. Pub. No. 20090084980, European Patent No. 2300983 B1, Int'l Pat. App. Pub. No. WO2014018584 A1, and Int'l Pat. App. Pub. No. WO2014018584 A1, each of which is incorporated herein by reference), confocal scanning microscopy, or other technologies comprising illumination schemes that illuminate (e.g., excite) only those query probe molecules near or on the surface of the solid support. Thus, in some embodiments, only query probes that are bound to an immobilized target near or on the surface produce a point-like emission signal (e.g., a “spot”) that can be confirmed as originating from a single molecule.

In some embodiments, the query probe comprises a fluorescent label having an emission wavelength. Detection of fluorescence emission at the emission wavelength of the fluorescent label indicates that the query probe is bound to an immobilized analyte. Binding of the query probe to the analyte is a “binding event”. In some embodiments of the technology, a binding event has a fluorescence emission having a measured intensity greater than a defined threshold. For example, in some embodiments a binding event has a fluorescence intensity that is above the background fluorescence intensity (e.g., the fluorescence intensity observed in the absence of an analyte). In some embodiments, a binding event has a fluorescence intensity that is at least 1, 2, 3, 4 or more standard deviations above the background fluorescence intensity (e.g., the fluorescence intensity observed in the absence of an analyte). In some embodiments, a binding event has a fluorescence intensity that is at least 2 standard deviations above the background fluorescence intensity (e.g., the fluorescence intensity observed in the absence of an analyte). In some embodiments, a binding event has a fluorescence intensity that is at least 1.5, 2, 3, 4, or 5 times the background fluorescence intensity (e.g., the mean fluorescence intensity observed in the absence of an analyte).

Accordingly, in some embodiments detecting fluorescence at the emission wavelength of the query probe that has an intensity above the defined threshold (e.g., at least 2 standard deviations greater than background intensity) indicates that a binding event has occurred (e.g., at a discrete location on the solid support where an analyte is immobilized). Also, in some embodiments detecting fluorescence at the emission wavelength of the query probe that has an intensity above the defined threshold (e.g., at least 2 standard deviations greater than background intensity) indicates that a binding event has started. Accordingly, in some embodiments detecting an absence of fluorescence at the emission wavelength of the query probe that has an intensity above the defined threshold (e.g., at least 2 standard deviations greater than background intensity) indicates that a binding event has ended (e.g., the query probe has dissociated from the analyte). The length of time between when the binding event started and when the binding event ended (e.g., the length of time that fluorescence at the emission wavelength of the fluorescent probe having an intensity above the defined threshold (e.g., at least 2 standard deviations greater than background intensity) is detected) is the dwell time of the binding event. A “transition” refers to the binding and dissociation of a query probe to the analyte (e.g., an on/off event), e.g., a query probe dissociating from a bound state or a query probe associating with an analyte from the unbound state.

Methods according to the technology comprise counting the number of query probe binding events that occur at each discrete location (e.g., at a position identified by x, y coordinates) on the solid support during a defined time interval that is the “acquisition time” (e.g., a time interval that is tens to hundreds to thousands of seconds, e.g., 5, 10, 15, 20, 25, 30, 35, 40, 45, 50, 55, or 60 seconds; e.g., 5, 10, 15, 20, 25, 30, 35, 40, 45, 50, 55, or 0 minutes; e.g., 1, 1.5, 2, 2.5, or 3 hours). In some embodiments, the acquisition time is approximately 1 to 10 seconds to 1 to 10 minutes (e.g., approximately 1 to 100 seconds, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 seconds, e.g., 1 to 100 minutes, e.g., 1, 2, 3, 4, 5, 6, 7, 8, 9, 10, 11, 12, 13, 14, 15, 16, 17, 18, 19, 20, 30, 40, 50, 60, 70, 80, 90, or 100 minutes).

Further, the length of time the query probe remains bound to the analyte during a binding event is the “dwell time” of the binding event. The number of binding events detected during the acquisition time and/or the lengths of the dwell times recorded for the binding events is/are characteristic of a query probe binding to an analyte and thus provide an indication that the analyte is immobilized at said discrete location and thus that the analyte is present in the sample.

Binding of the query probe to the immobilized analyte and/or and dissociation of the query probe from the immobilized analyte is/are monitored (e.g., using a light source to excite the fluorescent probe and detecting fluorescence emission from a bound query probe, e.g., using a fluorescence microscope) and/or recorded during a defined time interval (e.g., during the acquisition time). The number of times the query probe binds to the analyte during the acquisition time and/or the length of time the query probe remains bound to the analyte during each binding event and the length of time the query probe remains unbound to the analyte between each binding event (e.g., the “dwell times” in the bound and unbound states, respectively) are determined, e.g., by the use of a computer and software (e.g., to analyze the data using a hidden Markov model and Poisson statistics).

In some embodiments, positive and/or negative control samples are measured (e.g., a control sample known to comprise or not to comprise a target). Fluorescence detected in a negative control sample is “background fluorescence” or “background (fluorescence) intensity” or “baseline”.

In some embodiments, data comprising measurements of fluorescence intensity at the emission wavelength of the query probe are recorded as a function of time. In some embodiments, the number of binding events and the dwell times of binding events (e.g. for each immobilized analyte) are determined from the data (e.g., by determining the number of times and the lengths of time the fluorescence intensity is above a threshold background fluorescence intensity). In some embodiments, transitions (e.g., binding and dissociation of a query probe) are counted for each discrete location on the solid support where an analyte is immobilized. In some embodiments, a threshold number of transitions is used to discriminate the presence of an analyte at a discrete location on the solid support from background signal, non-analyte, and/or spurious binding of the query probe.

In some embodiments, a distribution of the number of transitions for each immobilized target is determined—e.g., the number of transitions is counted for each immobilized analyte observed. In some embodiments a histogram is produced. In some embodiments, characteristic parameters of the distribution are determined, e.g., the mean, median, peak, shape, etc. of the distribution are determined. In some embodiments, data and/or parameters (e.g., fluorescence data (e.g., fluorescence data in the time domain), kinetic data, characteristic parameters of the distribution, etc.) are analyzed by algorithms that recognize patterns and regularities in data, e.g., using artificial intelligence, pattern recognition, machine learning, statistical inference, neural nets, etc. In some embodiments, the analysis comprises use of a frequentist analysis and in some embodiments the analysis comprises use of a Bayesian analysis. In some embodiments, pattern recognition systems are trained using known “training” data (e.g., using supervised learning) and in some embodiments algorithms are used to discover previously unknown patterns (e.g., unsupervised learning). See, e.g., Duda, et al. (2001) Pattern classification (2nd edition), Wiley, New York; and Bishop (2006) Pattern Recognition and Machine Learning, Springer.

Pattern recognition (e.g., using training sets, supervised learning, unsupervised learning, and analysis of unknown samples) associates identified patterns with analytes such that particular patterns provide a “fingerprint” of particular analytes that find use in detection, quantification, and identification of analytes.

In some embodiments, the distribution produced from an analyte is significantly different than a distribution produced from a non-analyte or the distribution produced in the absence of an analyte. In some embodiments, a mean number of transitions is determined for the plurality of immobilized analytes. In some embodiments, the mean number of transitions observed for a sample comprising an analyte is approximately linearly related as a function of time and has a positive slope (e.g., the mean number of transitions increases approximately linearly as a function of time).

In some embodiments, the data are treated using statistics (e.g., Poisson statistics) to determine the probability of a transition occurring as a function of time at each discrete location on the solid support. In some particular embodiments, a relatively constant probability of a transition event occurring as a function of time at a discrete location on the solid support indicates the presence of an analyte at said discrete location on the solid support. In some embodiments, a correlation coefficient relating event number and elapsed time is calculated from the probability of a transition event occurring as a function of time at a discrete location on the solid support. In some embodiments, a correlation coefficient relating event number and elapsed time greater than 0.95 when calculated from the probability of a transition event occurring as a function of time at a discrete location on the solid support indicates the presence of an analyte at said discrete location on the solid support.

In some embodiments, dwell times of bound query probe (τₒₙ) and unbound query probe (τ_{off}) are used to identify the presence of an analyte in a sample and/or to distinguish a sample comprising an analyte from a sample comprising a non-analyte and/or not comprising the analyte. For example, the τₒₙ for an analyte is greater than the τₒₙ for a non-analyte; and, the τ_{off} for an analyte is smaller than the τ_{off} for a non-analyte. In some embodiments, measuring τₒₙ and τ_{off} for a negative control and for a sample indicates the presence or absence of the analyte in the sample. In some embodiments, a plurality of τₒₙ and τ_{off} values is determined for each of a plurality of spots imaged on a solid support, e.g., for a control (e.g., positive and/or negative control) and a sample suspected of comprising an analyte. In some embodiments, a mean τₒₙ and/or τ_{off} is determined for each of a plurality of spots imaged on a solid support, e.g., for a control (e.g., positive and/or negative control) and a sample suspected of comprising an analyte. In some embodiments, a plot of τₒₙ versus τ_{off} (e.g., mean Tₒₙ and τ_{off}, time-averaged τₒₙ and τ_{off}, etc.) for all imaged spots indicates the presence or absence of the analyte in the sample.

In some embodiments, the technology relates to use of SiMREPS assay conditions that are provided to modulate (e.g., increase and/or decrease) the association of query probes to analytes and/or to modulate (e.g., increase and/or decrease) the dissociation of query probes from analytes. In some embodiments, modulating (e.g., increasing and/or decreasing) the association of query probes to analytes and/or modulating (e.g., increasing and/or decreasing) the dissociation of query probes from analytes results in modulating (e.g., increasing and/or decreasing) the assay time (e.g., time required to collect signals indicating the kinetic activity of query probe transient interactions with analytes). In particular embodiments, assay time is decreased by increasing the rate of query probe association with analytes and/or increasing the rate of query probe dissociation from analytes. Exemplary assay conditions that are modulated to decrease assay time include, e.g., increasing the assay temperature (e.g., to a temperature above room temperature, (e.g., to 30° C. or more (e.g., to 30-37° C. (e.g., 30.0, 30.5, 31.0, 31.5, 32.0, 32.5, 33.0, 33.5, 34.0, 34.5, 35.0, 35.5, 36.0, 36.5, or 37.0), to 33-37 (e.g., 33.0, 33.5, 34.0, 34.5, 35.0, 35.5, 36.0, 36.5, or 37.0), or to more than 37° C.); by increasing the salt concentration (e.g., increasing salt from approximately 150 mM sodium ions to approximately 600 mM sodium ions); and/or by increasing the concentration of an organic solvent. In some embodiments, modulating (e.g., increasing and/or decreasing) the association of query probes to analytes and/or modulating (e.g., increasing and/or decreasing) the dissociation of query probes from analytes decreases the assay time by over 50% (e.g., by 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, or 90%).

In some embodiments, the technology detects analytes at a concentration in a composition (e.g., a sample) that is approximately 1 aM or more (e.g., approximately 1-10 aM (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 aM), approximately 10-100 aM (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 aM), or approximately 100-1000 aM (e.g., 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 aM).

In some embodiments, the technology detects analytes at a concentration in a composition (e.g., a sample) that is approximately 1 fM or more (e.g., approximately 1-10 fM (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 fM), approximately 10-100 fM (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fM), or approximately 100-1000 fM (e.g., 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 fM).

In some embodiments, the technology detects analytes at a concentration in a composition (e.g., a sample) that is approximately 1 pM or more (e.g., approximately 1-10 pM (e.g., 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 pM), approximately 10-100 pM (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 pM), or approximately 100-1000 pM (e.g., 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 pM).

For example, in some embodiments, the technology detects protein and/or nucleic acid analytes at concentrations of from approximately 50 aM to approximately 50 pM (e.g., the technology has a lower limit of detection, in some embodiments, of approximately 50 aM to approximately 50 pM). In some embodiments, the technology detects protein and/or nucleic acid analytes at concentrations of at least 10-20 aM, e.g., using embodiments of technologies as described herein (e.g., comprising use of a microfluidic device, nanoparticles, and/or irreversible linking of analytes to capture probes and/or to the imaging surface) that provide a capture efficiency of analytes of at least 50% to approximately 100% (e.g., at least 50, 55, 60, 65, 70, 75, 80, 85, 90, 95, 96, 97, 98, or 99% capture efficiency). That is, in some embodiments, technologies as described herein (e.g., comprising use of a microfluidic device, nanoparticles, and/or irreversible linking of analytes to capture probes and/or to the imaging surface) provide a capture efficiency of analytes that is at least 50% to approximately 100% (e.g., at least 50, 55, 60, 65, 70, 75, 80, 85, 90, 95, 96, 97, 98, or 99% capture efficiency) and thus provide a lower limit of detection of approximately 10-20 aM (e.g., 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0, 13.1, 13.2, 13.3, 13.4, 13.5, 13.6, 13.7, 13.8, 13.9, 14.0, 14.1, 14.2, 14.3, 14.4, 14.5, 14.6, 14.7, 14.8, 14.9, 15.0, 15.1, 15.2, 15.3, 15.4, 15.5, 15.6, 15.7, 15.8, 15.9, 16.0, 16.1, 16.2, 16.3, 16.4, 16.5, 16.6, 16.7, 16.8, 16.9, 17.0, 17.1, 17.2, 17.3, 17.4, 17.5, 17.6, 17.7, 17.8, 17.9, 18.0, 18.1, 18.2, 18.3, 18.4, 18.5, 18.6, 18.7, 18.8, 18.9, 19.0, 19.1, 19.2, 19.3, 19.4, 19.5, 19.6, 19.7, 19.8, 19.9, or 20.0 aM). See, e.g., Chang (2012) “Single molecule enzyme-linked immunosorbent assays: Theoretical Considerations” J. Immunological Methods 378: 102-115, incorporated herein by reference. In some embodiments, capture efficiency of analytes is at least 50% to approximately 100% (e.g., at least 50, 55, 60, 65, 70, 75, 80, 85, 90, 95, 96, 97, 98, or 99% capture efficiency) and at least 50% to approximately 100% (e.g., at least 50, 55, 60, 65, 70, 75, 80, 85, 90, 95, 96, 97, 98, or 99%) of the capture surface is imaged, which together provide a lower limit of detection for proteins and/or nucleic acids of approximately 0.05 aM to approximately 5 aM (e.g., a lower limit of detection of 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.00, 3.05, 3.10, 3.15, 3.20, 3.25, 3.30, 3.35, 3.40, 3.45, 3.50, 3.55, 3.60, 3.65, 3.70, 3.75, 3.80, 3.85, 3.90, 3.95, 4.00, 4.05, 4.10, 4.15, 4.20, 4.25, 4.30, 4.35, 4.40, 4.45, 4.50, 4.55, 4.60, 4.65, 4.70, 4.75, 4.80, 4.85, 4.90, 4.95, or 5.00 aM). Capabilities of the technology that are described using a lower limit of detection indicate that the technology detects analytes present at a concentration that is at least the lower limit of detection and, thus, the technology detects analytes that are present at a concentration that is higher than the lower limit of detection.

### Fluorescent Moieties

In some embodiments, a query probe and/or an analyte comprises a fluorescent moiety (e.g., a fluorogenic dye, also referred to as a “fluorophore” or a “fluor”). A wide variety of fluorescent moieties is known in the art and methods are known for linking a fluorescent moiety to analytes and/or query probes.

Examples of compounds that may be used as the fluorescent moiety include but are not limited to xanthene, anthracene, cyanine, porphyrin, and coumarin dyes. Examples of xanthene dyes that find use with the present technology include but are not limited to fluorescein, 6-carboxyfluorescein (6-FAM), 5-carboxyfluorescein (5-FAM), 5- or 6-carboxy-4, 7, 2′, 7′-tetrachlorofluorescein (TET), 5- or 6-carboxy-4′5′2′4′5′7′ hexachlorofluorescein (HEX), 5′ or 6′-carboxy-4′,5′-dichloro-2,′7′-dimethoxyfluorescein (JOE), 5-carboxy-2′,4′,5′,7′-tetrachlorofluorescein (ZOE), rhodol, rhodamine, tetramethylrhodamine (TAMRA), 4,7-dlchlorotetramethyl rhodamine (DTAMRA), rhodamine X (ROX), and Texas Red. Examples of cyanine dyes that may find use with the present invention include but are not limited to Cy 3, Cy 3B, Cy 3.5, Cy 5, Cy 5.5, Cy 7, and Cy 7.5. Other fluorescent moieties and/or dyes that find use with the present technology include but are not limited to energy transfer dyes, composite dyes, and other aromatic compounds that give fluorescent signals. In some embodiments, the fluorescent moiety comprises a quantum dot or polymer dot and polymeric dyes.

In some embodiments, the fluorescent moiety comprises a fluorescent protein (e.g., a green fluorescent protein (GFP), a modified derivative of GFP (e.g., a GFP comprising S65T, an enhanced GFP (e.g., comprising F64L)), or others known in the art such as, e.g., blue fluorescent protein (e.g., EBFP, EBFP2, Azurite, mKalamal), cyan fluorescent protein (e.g., ECFP, Cerulean, CyPet, mTurquoise2), and yellow fluorescent protein derivatives (e.g., YFP, Citrine, Venus, YPet). Embodiments provide that the fluorescent protein may be covalently or noncovalently bonded to one or more query probes, analytes, and/or capture probes.

Fluorescent dyes include, without limitation, d-Rhodamine acceptor dyes including Cy 5, dichloro[R110], dichloro[R6G], dichloro[TAMRA], dichloro[ROX] or the like, fluorescein donor dyes including fluorescein, 6-FAM, 5-FAM, or the like; Acridine including Acridine orange, Acridine yellow, Proflavin, pH 7, or the like; Aromatic Hydrocarbons including 2-Methylbenzoxazole, Ethyl p-dimethylaminobenzoate, Phenol, Pyrrole, benzene, toluene, or the like; Arylmethine Dyes including Auramine O, Crystal violet, Crystal violet, glycerol, Malachite Green or the like; Coumarin dyes including 7-Methoxycoumarin-4-acetic acid, Coumarin 1, Coumarin 30, Coumarin 314, Coumarin 343, Coumarin 6 or the like; Cyanine Dyes including 1,1′-diethyl-2,2′-cyanine iodide, Cryptocyanine, Indocarbocyanine (C3) dye, Indodicarbocyanine (C5) dye, Indotricarbocyanine (C7) dye, Oxacarbocyanine (C3) dye, Oxadicarbocyanine (C5) dye, Oxatricarbocyanine (C7) dye, Pinacyanol iodide, Stains all, Thiacarbocyanine (C3) dye, ethanol, Thiacarbocyanine (C3) dye, n-propanol, Thiadicarbocyanine (C5) dye, Thiatricarbocyanine (C7) dye, or the like; Dipyrrin dyes including N,N′-Difluoroboryl-1,9-dimethyl-5-(4-iodophenyl)-dipyrrin, N,N′-Difluoroboryl-1,9-dimethyl-5-1(4-(2-trimethylsilylethynyl), N,N′-Difluoroboryl-1,9-dimethyl-5-phenydipyrrin, or the like; Merocyanines including 4-(dicyanomethylene)-2-methyl-6-(p-dimethylaminostyryl)-4H-pyran (DCM), acetonitrile, 4-(dicyanomethylene)-2-methyl-6-(p-dimethylaminostyryl)-4H-pyran (DCM), methanol, 4-Dimethylamino-4′-nitrostilbene, Merocyanine 540, or the like; Miscellaneous Dyes including 4′,6-Diamidino-2-phenylindole (DAPI), dimethylsulfoxide, 7-Benzylamino-4-nitrobenz-2-oxa-1,3-diazole, Dansyl glycine, Dansyl glycine, dioxane, Hoechst 33258, DMF, Hoechst 33258, Lucifer yellow CH, Piroxicam, Quinine sulfate, Quinine sulfate, Squarylium dye III, or the like; Oligophenylenes including 2,5-Diphenyloxazole (PPO), Biphenyl, POPOP, p-Quaterphenyl, p-Terphenyl, or the like; Oxazines including Cresyl violet perchlorate, Nile Blue, methanol, Nile Red, ethanol, Oxazine 1, Oxazine 170, or the like; Polycyclic Aromatic Hydrocarbons including 9,10-Bis(phenylethynyl)anthracene, 9,10-Diphenylanthracene, Anthracene, Naphthalene, Perylene, Pyrene, or the like; polyene/polyynes including 1,2-diphenylacetylene, 1,4-diphenylbutadiene, 1,4-diphenylbutadiyne, 1,6-Diphenylhexatriene, Beta-carotene, Stilbene, or the like; Redox-active Chromophores including Anthraquinone, Azobenzene, Benzoquinone, Ferrocene, Riboflavin, Tris(2,2′-bipyridypruthenium(II), Tetrapyrrole, Bilirubin, Chlorophyll a, diethyl ether, Chlorophyll a, methanol, Chlorophyll b, Diprotonated-tetraphenylporphyrin, Hematin, Magnesium octaethylporphyrin, Magnesium octaethylporphyrin (MgOEP), Magnesium phthalocyanine (MgPc), PrOH, Magnesium phthalocyanine (MgPc), pyridine, Magnesium tetramesitylporphyrin (MgTMP), Magnesium tetraphenylporphyrin (MgTPP), Octaethylporphyrin, Phthalocyanine (Pc), Porphin, ROX, TAMRA, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), Tetraphenylporphyrin (TPP), Vitamin B12, Zinc octaethylporphyrin (ZnOEP), Zinc phthalocyanine (ZnPc), pyridine, Zinc tetramesitylporphyrin (ZnTMP), Zinc tetramesitylporphyrin radical cation, Zinc tetraphenylporphyrin (ZnTPP), or the like; Xanthenes including Eosin Y, Fluorescein, basic ethanol, Fluorescein, ethanol, Rhodamine 123, Rhodamine 6G, Rhodamine B, Rose bengal, Sulforhodamine 101, or the like; or mixtures or combination thereof or synthetic derivatives thereof.

Several classes of fluorogenic dyes and specific compounds are known that are appropriate for particular embodiments of the technology: xanthene derivatives such as fluorescein, rhodamine, Oregon green, eosin, and Texas red; cyanine derivatives such as cyanine, indocarbocyanine, oxacarbocyanine, thiacarbocyanine, and merocyanine; naphthalene derivatives (dansyl and prodan derivatives); coumarin derivatives; oxadiazole derivatives such as pyridyloxazole, nitrobenzoxadiazole, and benzoxadiazole; pyrene derivatives such as cascade blue; oxazine derivatives such as Nile red, Nile blue, cresyl violet, and oxazine 170; acridine derivatives such as proflavin, acridine orange, and acridine yellow; arylmethine derivatives such as auramine, crystal violet, and malachite green; and tetrapyrrole derivatives such as porphin, phtalocyanine, bilirubin. In some embodiments the fluorescent moiety a dye that is xanthene, fluorescein, rhodamine, BODIPY, cyanine, coumarin, pyrene, phthalocyanine, phycobiliprotein, ALEXA FLUOR® 350, ALEXA FLUOR® 405, ALEXA FLUOR® 430, ALEXA FLUOR® 488, ALEXA FLUOR® 514, ALEXA FLUOR® 532, ALEXA FLUOR® 546, ALEXA FLUOR® 555, ALEXA FLUOR® 568, ALEXA FLUOR® 568, ALEXA FLUOR® 594, ALEXA FLUOR® 610, ALEXA FLUOR® 633, ALEXA FLUOR® 647, ALEXA FLUOR® 660, ALEXA FLUOR® 680, ALEXA FLUOR® 700, ALEXA FLUOR® 750, or a squaraine dye. In some embodiments, the label is a fluorescently detectable moiety as described in, e.g., Haugland (September 2005) MOLECULAR PROBES HANDBOOK OF FLUORESCENT PROBES AND RESEARCH CHEMICALS (10th ed.), which is herein incorporated by reference in its entirety.

In some embodiments the label (e.g., a fluorescently detectable label) is one available from ATTO-TEC GmbH (Am Eichenhang 50, 57076 Siegen, Germany), e.g., as described in U.S. Pat. Appl. Pub. Nos. 20110223677, 20110190486, 20110172420, 20060179585, and 20030003486; and in U.S. Pat. No. 7,935,822, all of which are incorporated herein by reference (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rhol01, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740).

One of ordinary skill in the art will recognize that dyes having emission maxima outside these ranges may be used as well. In some cases, dyes ranging between 500 nm to 700 nm have the advantage of being in the visible spectrum and can be detected using existing photomultiplier tubes. In some embodiments, the broad range of available dyes allows selection of dye sets that have emission wavelengths that are spread across the detection range. Detection systems capable of distinguishing many dyes are known in the art.

### Methods

Some embodiments provide a method of identifying an analyte by repetitive query probe binding. In some embodiments, methods comprise immobilizing an analyte to a solid support. In some embodiments, the solid support is a surface (e.g., a substantially planar surface, a rounded surface), e.g., a surface in contact with a bulk solution, e.g., a bulk solution comprising analyte. In some embodiments, the solid support is a freely diffusible solid support (e.g., a bead, a colloidal particle, e.g., a colloidal particle having a diameter of approximately 10-1000 nm (e.g., 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, or 1000 nm)), e.g., that freely diffuses within the bulk solution, e.g., a bulk solution comprising the analyte. In some embodiments, immobilizing an analyte to a solid support comprises covalent interaction between the solid support and analyte. In some embodiments, immobilizing an analyte to a solid support comprises non-covalent interaction between the solid support and analyte. In some embodiments, the analyte (e.g., a molecule, e.g., a molecule such as, e.g., a protein, peptide, nucleic acid, small molecule, lipid, metabolite, drug, etc.) is stably immobilized to a surface and methods comprise repetitive (e.g., transient, low-affinity) binding of a query probe to the analyte. In some embodiments, methods comprise detecting the repetitive (e.g., transient, low-affinity) binding of a query probe to the analyte. In some embodiments, methods comprise generating a dataset comprising a signal produced from query probe binding to the analyte (e.g., a dataset of query probe signal as a function of time) and information (e.g., coordinates, e.g., x, y coordinates) describing the spatial position on the surface of the query probe binding to the analyte. In some embodiments, the dataset is processed (e.g., manipulated, transformed, visualized, etc.), e.g., to improve the spatial resolution of the query probe binding events. For example, in particular embodiments, the dataset (e.g., comprising query probe signal as a function of time and information (e.g., coordinates, e.g., x, y coordinates) describing the spatial position on the surface of the query probe binding to the analyte) is subjected to processing. In some embodiments, the processing comprises a frame-by-frame subtraction process to generate differential intensity profiles showing query probe binding or dissociation events within each frame of the time series data. Data collected during the development of the technology described herein indicate that the differential intensity profiles have a higher resolution than the query probe binding signal vs. position map. In some embodiments, after determining the spatial position (e.g., x, y coordinates) of each query probe binding and/or dissociation event, a plurality of events is clustered according to spatial position and the kinetics of the events within each cluster are subjected to statistical analysis to determine whether the cluster of events originates from a given analyte.

For instance, some embodiments of methods for quantifying one or more surface-immobilized or diffusing analytes comprise one or more steps including, e.g., measuring the signal of one or more transiently binding query probes to the immobilized analyte(s) with single-molecule sensitivity. In some embodiments, methods comprise tracking (e.g., detecting and/or recording the position of) analytes independently from query probe binding. In some embodiments, the methods further comprise calculating the time-dependent probe binding signal intensity changes at the surface as a function of position (e.g., x, y position). In some embodiments, calculating the time-dependent query probe binding signal intensity changes at the surface as a function of position (e.g., x, y position) produces a “differential intensity profile” for query probe binding to the analyte. In some embodiments, the methods comprise determining the position (e.g., x, y position) of each query probe binding and dissociation event (“event”) with sub-pixel accuracy from a differential intensity profile. In some embodiments, methods comprise grouping events into local clusters by position (e.g., x, y position) on the surface, e.g., to associate events for a single immobilized analyte. In some embodiments, the methods comprise calculating kinetic parameters from each local cluster of events to determine whether the cluster originates from a particular analyte, e.g., from transient probe binding to a particular analyte.

Embodiments of methods are not limited in the analyte that is detected. For example, in some embodiments the analyte is polypeptide, e.g., a protein or a peptide. In some embodiments, the analyte is a nucleic acid. In some embodiments, the analyte is a small molecule.

In some embodiments, the interaction between the analyte and the query probe is distinguishably influenced by a covalent modification of the analyte. For example, in some embodiments, the analyte is a polypeptide comprising a post-translational modification, e.g., a protein or a peptide comprising a post-translational modification. In some embodiments, a post-translational modification of a polypeptide affects the transient binding of a query probe with the analyte, e.g., the query probe signal is a function of the presence or absence of the post-translational modification on the polypeptide. For example, in some embodiments, the analyte is a nucleic acid comprising an epigenetic modification, e.g., a nucleic acid comprising a methylated base. In some embodiments, the analyte is a nucleic acid comprising a covalent modification to a nucleobase, a ribose, or a deoxyribose moiety of the analyte.

In some embodiments, a modification of a nucleic acid affects the transient binding of a query probe with the analyte, e.g., the query probe signal is a function of the presence or absence of the modification on the nucleic acid.

In some embodiments, the transient interaction between the post-translational modification and the query probe is mediated by a chemical affinity tag, e.g., a chemical affinity tag comprising a nucleic acid.

In some embodiments, the query probe is a nucleic acid or an aptamer. In some embodiments, the query probe is a low-affinity antibody, antibody fragment, or nanobody.

In some embodiments, the query probe is a DNA-binding protein, RNA-binding protein, or a DNA-binding ribonucleoprotein complex.

In some embodiments, the position, e.g., the (x,y) position, of each binding or dissociation event is determined by subjecting the differential intensity profile to centroid determination, least-squares fitting to a Gaussian function, least-square fitting to an airy disk function, least-squares fitting to a polynomial function (e.g., a parabola), or maximum likelihood estimation.

In some embodiments, the capture probe is a high-affinity antibody, antibody fragment, or nanobody. In some embodiments, the capture probe is a nucleic acid. In some embodiments, capture is mediated by a covalent bond cross-linking the analyte to the surface. In some embodiments, the analyte is subjected to thermal denaturation in the presence of a carrier prior to surface immobilization. In some embodiments, the analyte is subjected to chemical denaturation in the presence of a carrier prior to surface immobilization, e.g., the analyte is denatured with a denaturant such as urea, formamide, guanidinium chloride, high ionic strength, low ionic strength, high pH, low pH, or sodium dodecyl sulfate (SDS).

### Systems

Embodiments of the technology relate to systems for detecting analytes. For example, in some embodiments, the technology provides a system for quantifying one or more analytes, wherein the system comprises a surface-bound capture probe or a surface-bound moiety that stably binds the analyte. In some embodiments, the surface-bound capture probe or the surface-bound moiety stably binds the analyte via a binding site, a epitope, or a recognition site (e.g., a first binding site, a first epitope, or a first recognition site). In some embodiments, systems further comprise a query probe that binds the analyte with a low affinity at a second binding site, a second epitope, or a second recognition site. In some embodiments, the query probe is freely diffusible in the bulk solution contacting the surface of the system. Furthermore, some system embodiments comprise a detection component that records a signal from the interaction of the query probe with the analyte. For example, in some embodiments the detection component records the change in the signal as a function of time produced from the interaction of the query probe with the analyte. In some embodiments, the detection component records the spatial position (e.g., as an x, y coordinate pair) and intensity of binding and dissociation events of the query probe to and from said analyte. In some embodiments, the detection component records the spatial position (e.g., as an x, y coordinate pair) and the beginning and/or ending time of binding and dissociation events of the query probe to and from said analyte. In some embodiments, the detection component records the spatial position (e.g., as an x, y coordinate pair) and the length of time of binding and dissociation events of the query probe to and from said analyte.

System embodiments comprise analytical processes (e.g., embodied in a set of instructions, e.g., encoded in software, that direct a microprocessor to perform the analytical processes) to identify an individual molecule of the analyte. In some embodiments, analytical processes use the spatial position data and timing (e.g., start, end, or length of time) of repeated binding and dissociation events to said analyte as input data.

Embodiments of systems are not limited in the analyte that is detected. For example, in some embodiments the analyte is polypeptide, e.g., a protein or a peptide. In some embodiments, the analyte is a nucleic acid. In some embodiments, the analyte is a small molecule.

In some embodiments, the interaction between the analyte and the query probe is distinguishably influenced by a covalent modification of the analyte. For example, in some embodiments, the analyte is a polypeptide comprising a post-translational modification, e.g., a protein or a peptide comprising a post-translational modification. In some embodiments, a post-translational modification of a polypeptide affects the transient binding of a query probe with the analyte, e.g., the query probe signal is a function of the presence or absence of the post-translational modification on the polypeptide. For example, in some embodiments, the analyte is a nucleic acid comprising an epigenetic modification, e.g., a nucleic acid comprising a methylated base. In some embodiments, a modification of a nucleic acid affects the transient binding of a query probe with the analyte, e.g., the query probe signal is a function of the presence or absence of the modification on the nucleic acid.

In some embodiments, the transient interaction between the post-translational modification and the query probe is mediated by a chemical affinity tag, e.g., a chemical affinity tag comprising a nucleic acid.

In some embodiments, the query probe is a nucleic acid or an aptamer. In some embodiments, the query probe is a low-affinity antibody, antibody fragment, or nanobody. In some embodiments, the query probe is a DNA-binding protein, RNA-binding protein, or a DNA-binding ribonucleoprotein complex.

In some embodiments, the analyte is a nucleic acid comprising a covalent modification to a nucleobase, a ribose, or a deoxyribose moiety of the analyte.

In some embodiments, the capture probe is a high-affinity antibody, antibody fragment, or nanobody. In some embodiments, the capture probe is a nucleic acid. In some embodiments, capture is mediated by a covalent bond cross-linking the analyte to the surface. In some embodiments, the analyte is subjected to thermal denaturation in the presence of a carrier prior to surface immobilization. In some embodiments, the analyte is subjected to chemical denaturation in the presence of a carrier prior to surface immobilization, e.g., the analyte is denatured with a denaturant such as urea, formamide, guanidinium chloride, high ionic strength, low ionic strength, high pH, low pH, or sodium dodecyl sulfate (SDS).

Some system embodiments of the technology comprise components for the detection and quantification of an analyte. Systems according to the technology comprise, e.g., a solid support (e.g., a microscope slide, a coverslip, an avidin (e.g., streptavidin)-conjugated microscope slide or coverslip, a solid support comprising a zero mode waveguide array, or the like), and a query probe as described herein.

Some system embodiments comprise a detection component that is a fluorescence microscope comprising an illumination configuration to excite bound query probes (e.g., a prism-type total internal reflection fluorescence (TIRF) microscope, an objective-type TIRF microscope, a near-TIRF or HiLo microscope, a confocal laser scanning microscope, a zero-mode waveguide, and/or an illumination configuration capable of parallel monitoring of a large area of the slide or coverslip (>100 μm²) while restricting illumination to a small region of space near the surface). Some embodiments comprise a fluorescence detector, e.g., a detector comprising an intensified charge coupled device (ICCD), an electron-multiplying charge coupled device (EM-CCD), a complementary metal-oxide-semiconductor (CMOS), a photomultiplier tube (PMT), an avalanche photodiode (APD), and/or another detector capable of detecting fluorescence emission from single chromophores. Some particular embodiments comprise a component configured for lens-free imaging, e.g., a lens-free microscope, e.g., a detection and/or imaging component for directly imaging on a detector (e.g., a CMOS) without using a lens.

Some embodiments comprise a computer and software encoding instructions for the computer to perform, e.g., to control data acquisition and/or analytical processes for processing data.

Some embodiments comprise optics, such as lenses, mirrors, dichroic mirrors, optical filters, etc., e.g., to detect fluorescence selectively within a specific range of wavelengths or multiple ranges of wavelengths.

For example, in some embodiments, computer-based analysis software is used to translate the raw data generated by the detection assay (e.g., the presence, absence, or amount of one or more analytes, e.g., as a function time and/or position (e.g., x, y coordinates) on the surface) into data of predictive value for a clinician. The clinician can access the predictive data using any suitable means.

Some system embodiments comprise a computer system upon which embodiments of the present technology may be implemented. In various embodiments, a computer system includes a bus or other communication mechanism for communicating information and a processor coupled with the bus for processing information. In various embodiments, the computer system includes a memory, which can be a random-access memory (RAM) or other dynamic storage device, coupled to the bus, and instructions to be executed by the processor. Memory also can be used for storing temporary variables or other intermediate information during execution of instructions to be executed by the processor. In various embodiments, the computer system can further include a read only memory (ROM) or other static storage device coupled to the bus for storing static information and instructions for the processor. A storage device, such as a magnetic disk or optical disk, can be provided and coupled to the bus for storing information and instructions.

In various embodiments, the computer system is coupled via the bus to a display, such as a cathode ray tube (CRT) or a liquid crystal display (LCD), for displaying information to a computer user. An input device, including alphanumeric and other keys, can be coupled to the bus for communicating information and command selections to the processor. Another type of user input device is a cursor control, such as a mouse, a trackball, or cursor direction keys for communicating direction information and command selections to the processor and for controlling cursor movement on the display. This input device typically has two degrees of freedom in two axes, a first axis (e.g., x) and a second axis (e.g., y), that allows the device to specify positions in a plane.

A computer system can perform embodiments of the present technology. Consistent with certain implementations of the present technology, results can be provided by the computer system in response to the processor executing one or more sequences of one or more instructions contained in the memory. Such instructions can be read into the memory from another computer-readable medium, such as a storage device. Execution of the sequences of instructions contained in the memory can cause the processor to perform the methods described herein. Alternatively, hard-wired circuitry can be used in place of or in combination with software instructions to implement the present teachings. Thus, implementations of the present technology are not limited to any specific combination of hardware circuitry and software.

In some embodiments, steps of the described methods are implemented in software code, e.g., a series of procedural steps instructing a computer and/or a microprocessor to produce and/or transform data as described above. In some embodiments, software instructions are encoded in a programming language such as, e.g., BASIC, NeXTSTEP, C, C++, C#, Objective C, Java, MATLAB, Mathematica, Perl, PHP, Ruby, Scala, Lisp, Smalltalk, Python, Swift, or R.

In some embodiments, one or more steps or components are provided in individual software objects connected in a modular system. In some embodiments, the software objects are extensible and portable. In some embodiments, the objects comprise data structures and operations that transform the object data. In some embodiments, the objects are used by manipulating their data and invoking their methods. Accordingly, embodiments provide software objects that imitate, model, or provide concrete entities, e.g., for numbers, shapes, data structures, that are manipulable. In some embodiments, software objects are operational in a computer or in a microprocessor. In some embodiments, software objects are stored on a computer readable medium.

In some embodiments, a step of a method described herein is provided as an object method. In some embodiments, data and/or a data structure described herein is provided as an object data structure.

Some embodiments provide an object-oriented pipeline for processing data, e.g., comprising one or more software objects, to produce a result.

Embodiments comprise use of code that produces and manipulates software objects, e.g., as encoded using a language such as but not limited to Java, C++, C#, Python, PHP, Ruby, Perl, Object Pascal, Objective-C, Swift, Scala, Common Lisp, and Smalltalk.

The term “computer-readable medium” as used herein refers to any media that participates in providing instructions to the processor for execution. Such a medium can take many forms, including but not limited to, non-volatile media, volatile media, and transmission media. Examples of non-volatile media can include, but are not limited to, optical or magnetic disks, such as a storage device. Examples of volatile media can include, but are not limited to, dynamic memory. Examples of transmission media can include, but are not limited to, coaxial cables, copper wire, and fiber optics, including the wires that comprise the bus.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

Various forms of computer readable media can be involved in carrying one or more sequences of one or more instructions to the processor for execution. For example, the instructions can initially be carried on the magnetic disk of a remote computer. The remote computer can load the instructions into its dynamic memory and send the instructions over a network connection (e.g., a LAN, a WAN, the internet, a telephone line). A local computer system can receive the data and transmit it to the bus. The bus can carry the data to the memory, from which the processor retrieves and executes the instructions. The instructions received by the memory may optionally be stored on a storage device either before or after execution by the processor.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

In accordance with such a computer system, some embodiments of the technology provided herein further comprise functionalities for collecting, storing, and/or analyzing data (e.g., presence, absence, concentration of an analyte). For example, some embodiments contemplate a system that comprises a processor, a memory, and/or a database for, e.g., storing and executing instructions, analyzing fluorescence, image data, performing calculations using the data, transforming the data, and storing the data. It some embodiments, an algorithm applies a statistical model (e.g., a Poisson model or hidden Markov model) to the data.

In some embodiments, systems comprise a computer and/or data storage provided virtually (e.g., as a cloud computing resource). In particular embodiments, the technology comprises use of cloud computing to provide a virtual computer system that comprises the components and/or performs the functions of a computer as described herein. Thus, in some embodiments, cloud computing provides infrastructure, applications, and software as described herein through a network and/or over the internet. In some embodiments, computing resources (e.g., data analysis, calculation, data storage, application programs, file storage, etc.) are remotely provided over a network (e.g., the internet), typically through a web browser. For example, many web browsers are capable of running applications, which can themselves be application programming interfaces (“API's”) to more sophisticated applications running on remote servers. In some embodiments, cloud computing involves using a web browser interface to control an application program that is running on a remote server.

Many diagnostics involve determining the presence of, or a nucleotide sequence of, one or more nucleic acids.

In some embodiments, an equation comprising variables representing the presence, absence, concentration, amount, or sequence properties of one or more analytes produces a value that finds use in making a diagnosis or assessing the presence or qualities of an analyte. As such, in some embodiments this value is presented by a device, e.g., by an indicator related to the result (e.g., an LED, an icon on a display, a sound, or the like). In some embodiments, a device stores the value, transmits the value, or uses the value for additional calculations. In some embodiments, an equation comprises variables representing the presence, absence, concentration, amount, or properties of one or more analytes.

Thus, in some embodiments, the present technology provides the further benefit that a clinician, who is not likely to be trained in analytical assays, need not understand the raw data. The data are presented directly to the clinician in its most useful form. The clinician is then able to utilize the information to optimize the care of a subject. The present technology contemplates any method capable of receiving, processing, and transmitting the information to and from laboratories conducting the assays, information providers, medical personal, and/or subjects. For example, in some embodiments of the present technology, a sample is obtained from a subject and submitted to a profiling service (e.g., a clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center or subjects may collect the sample themselves and directly send it to a profiling center. Where the sample comprises previously determined biological information, the information may be directly sent to the profiling service by the subject (e.g., an information card containing the information may be scanned by a computer and the data transmitted to a computer of the profiling center using electronic communication systems). Once received by the profiling service, the sample is processed and a profile is produced that is specific for the diagnostic or prognostic information desired for the subject. The profile data are then prepared in a format suitable for interpretation by a treating clinician. For example, rather than providing raw expression data, the prepared format may represent a diagnosis or risk assessment for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor. In some embodiments, the information is first analyzed at the point of care or at a regional facility. The raw data are then sent to a central processing facility for further analysis and/or to convert the raw data to information useful for a clinician or patient. The central processing facility provides the advantage of privacy (all data are stored in a central facility with uniform security protocols), speed, and uniformity of data analysis. The central processing facility can then control the fate of the data following treatment of the subject. For example, using an electronic communication system, the central facility can provide data to the clinician, the subject, or researchers. In some embodiments, the subject is able to access the data using the electronic communication system. The subject may chose further intervention or counseling based on the results. In some embodiments, the data are used for research use. For example, the data may be used to further optimize the inclusion or elimination of markers as useful indicators of a particular condition associated with the disease.

### Samples

In some embodiments, analytes are isolated from a biological sample. Analytes can be obtained from any material (e.g., cellular material (live or dead), extracellular material, viral material, environmental samples (e.g., metagenomic samples), synthetic material (e.g., amplicons such as provided by PCR or other amplification technologies)), obtained from an animal, plant, bacterium, archaeon, fungus, or any other organism. Biological samples for use in the present technology include viral particles or preparations thereof. Analytes can be obtained directly from an organism or from a biological sample obtained from an organism, e.g., from blood, urine, cerebrospinal fluid, seminal fluid, saliva, sputum, stool, hair, sweat, tears, skin, and tissue. Exemplary samples include, but are not limited to, whole blood, lymphatic fluid, serum, plasma, buccal cells, sweat, tears, saliva, sputum, hair, skin, biopsy, cerebrospinal fluid (CSF), amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs, aspirates (e.g., bone marrow, fine needle, etc.), washes (e.g., oral, nasopharyngeal, bronchial, bronchialalveolar, optic, rectal, intestinal, vaginal, epidermal, etc.), breath condensate, and/or other specimens.

Any tissue or body fluid specimen may be used as a source of analytes for use in the technology, including forensic specimens, archived specimens, preserved specimens, and/or specimens stored for long periods of time, e.g., fresh-frozen, methanol/acetic acid fixed, or formalin-fixed paraffin embedded (FFPE) specimens and samples. Analytes can also be isolated from cultured cells, such as a primary cell culture or a cell line. The cells or tissues from which analytes are obtained can be infected with a virus or other intracellular pathogen. A sample can also be total RNA extracted from a biological specimen, a cDNA library, viral, or genomic DNA. A sample may also be isolated DNA from a non-cellular origin, e.g. amplified/isolated DNA that has been stored in a freezer.

Analytes (e.g., nucleic acid molecules, polypeptides, lipids) can be obtained, e.g., by extraction from a biological sample, e.g., by a variety of techniques such as those described by Maniatis, et al. (1982) Molecular Cloning: A Laboratory Manual, Cold Spring Harbor, N.Y. (see, e.g., pp. 280-281).

In some embodiments, the technology provides for the size selection of analytes, e.g., to provide a defined size range of molecules including the analytes.

### Uses

Various embodiments relate to the detection of a wide range of analytes. For example, in some embodiments the technology finds use in detecting a nucleic acid (e.g., a DNA or RNA). In some embodiments, the technology finds use in detecting a nucleic acid comprising a particular target sequence. In some embodiments, the technology finds use in detecting a nucleic acid comprising a particular mutation (e.g., a single nucleotide polymorphism, an insertion, a deletion, a missense mutation, a nonsense mutation, a genetic rearrangement, a gene fusion, etc.). In some embodiments, the technology finds use in detection a polypeptide (e.g., a protein, a peptide). In some embodiments, the technology finds use in detecting a polypeptide encoded by a nucleic acid comprising a mutation (e.g., a polypeptide comprising a substitution, a truncated polypeptide, a mutant or variant polypeptide).

In some embodiments, the technology finds use in detecting post-translational modifications to polypeptides (e.g., phosphorylation, methylation, acetylation, glycosylation (e.g., O-linked glycosylation, N-linked glycosylation, ubiquitination, attachment of a functional group (e.g., myristoylation, palmitoylation, isoprenylation, prenylation, farnesylation, geranylation, geranylgeranylation, glypiation, glycosylphosphatidylinositol (GPI) anchor formation), hydroxylation, biotinylation, pegylation, oxidation, SUMOylation, disulfide bridge formation, disulfide bridge cleavage, proteolytic cleavage, amidation, sulfation, pyrrolidone carboxylic acid formation. In some embodiments, the technology finds use in the detection of the loss of these features, e.g., dephosporylation, demethylation, de acetylation, de glycosylation, deamidation, dehydroxylation, deubiquitination, etc. In some embodiments, the technology finds use in detecting epigenetic modifications to DNA or RNA (e.g., methylation (e.g., methylation of CpG sites), hydroxymethylation). In some embodiments, the technology finds use in detecting the loss of these features, e.g., demethylation of DNA or RNA, etc. In some embodiments, the technology finds use in detecting alterations in chromatin structure, nucleosome structure, histone modification, etc., and in detecting damage to nucleic acids.

In some embodiments, the technology finds use as a molecular diagnostic assay, e.g., to assay samples having small specimen volumes (e.g., a droplet of blood, e.g., for mail-in service). In some embodiments, the technology provides for the early detection of cancer or infectious disease using sensitive detection of very low-abundance analyte biomarkers. In some embodiments, the technology finds use in molecular diagnostics to assay epigenetic modifications of protein biomarkers (e.g., post-translational modifications).

In some embodiments, the technology finds use in characterizing multimolecular complexes (e.g., characterizing one or more components of a multimolecular complex), e.g., a multiprotein complex, a nucleic acid/protein complex, a molecular machine, an organelle (e.g., a cell-free mitochondrion, e.g., in plasma), cell, virus particle, organism, tissue, or any macromolecular structure or entity that can be captured and is amenable to analysis by the technology described herein. For example, in some embodiments a multimolecular complex is isolated and the technology finds use in characterizing, identifying, quantifying, and/or detecting one or more molecules (analytes) associated with the multimolecular complex. In some embodiments an extracellular vesicle is isolated and the technology finds use in characterizing, identifying, quantifying, and/or detecting one or more molecules (analytes) associated with the vesicle. In some embodiments, the technology finds use in characterizing, identifying, quantifying, and/or detecting a protein (e.g., a surface protein) and/or an analytes present inside the vesicle, e.g., a protein, nucleic acid, or other analyte described herein. In some embodiments, the vesicle is fixed and permeabilized prior to analysis.

### Analyte Capture by Nanoparticles Comprising Capture Probes

In some embodiments, the technology relates to using nanoparticles to capture analytes for analysis by SiMREPS. In some embodiments, the technology comprises use of nanoparticles having a diameter ranging from approximately 5 nanometers to approximately 200 nanometers (e.g., approximately 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 nanometers) and comprising (e.g., coated with, linked to) a capture probe (e.g., nucleic acid, antibody, antigen) having a specific affinity for an analyte (e.g., nucleic acid, antigen, antibody, respectively). In some embodiments, the nanoparticles have a diameter that is large enough to be collected (e.g., deposited (e.g., immobilized)) efficiently at the surface and small enough to fit entirely or mostly within an excitation field (e.g., a TIRF evanescent field). Furthermore, in some embodiments, the technology comprises use of a nanoparticle having a diameter less than approximately 200 nm to reduces scattering of excitation or emission light, thus increasing the sensitivity of detecting single binding events of the query probe.

After capture of analytes by the nanoparticles (e.g., by capture probes of the nanoparticles), nanoparticles are collected (e.g., deposition (e.g., immobilized)) at a surface for SiMREPS analysis, e.g., using single-molecule imaging (e.g., total internal reflection fluorescence, TIRF) in the presence of a query probe that repeatedly binds to the captured analyte to provide a detectable signal that distinguishes between specific binding of the query probe to the analyte and nonspecific binding, if any, of the query probe to other non-analyte entities.

According to embodiments of the technology, the nanoparticles are separable from the surrounding medium by the application of an external force, e.g., to collect (e.g., deposit (e.g., immobilize)) the nanoparticles comprising the analyte at a surface. The technology is not limited in the method (e.g., external force) used to collect (e.g., deposit (e.g., immobilize)) nanoparticles on a surface. For example, in some embodiments, nanoparticles are deposited by providing a magnetic field (e.g., for magnetic or paramagnetic nanoparticles), by providing an electrical field (e.g., for polar and/or electrically charged nanoparticles), and/or by providing an inertial force (e.g., centrifugation (e.g., for nanoparticles that have different density than the surrounding medium) and/or gravity). In an exemplary embodiment, a suspension of super-paramagnetic nanoparticles is used to capture an analyte from solution and the nanoparticles comprising the analyte are rapidly (e.g., in less than 5 minutes (e.g., least than 5, 4.5, 4, 3.5, 3, 2.5, 2, 1.5, 1, or 0.5 minutes)) deposited onto a glass coverslip with an external rare earth magnet for SiMREPS analysis. In another exemplary embodiment, a suspension of gold nanoparticles is used to capture an analyte from solution and then the nanoparticles comprising the analyte are deposited by centrifugation onto a glass coverslip for SiMREPS analysis.

In some embodiments, the use of nanoparticles with the SiMREPS technology increases the speed of analyte capture and/or increases the efficiency of antigen capture relative to capture by diffusion alone. Accordingly, use of nanoparticles with SiMREPS decreases the time-to-result and/or increases the sensitivity of SiMREPS assays.

The terms “paramagnetic” and “superparamagnetic” as used herein are interchangeable. Paramagnetic and superparamagnetic materials (e.g., when fabricated as nanoparticles) have the property of responding to an external magnetic field when present, but dissipating any residual magnetism immediately upon release of the external magnetic field, and are thus easily resuspended and remain monodisperse, but when placed in proximity to a magnetic field, clump tightly, the process being fully reversible by simply removing the magnetic field.

As used herein, the term “magnetic force field” or “magnetic field” refers to a volume defined by the magnetic flux lines between two poles of a magnet or two faces of a coil. Electromagnets and driving circuitry can be used to generate magnetic fields and localized magnetic fields. Permanent magnets may also be used. Preferred permanent magnetic materials include NdFeB (Neodymium-Iron-Boron Nd₂Fe₁₄B), Ferrite (Strontium or Barium Ferrite), AlNiCo (Aluminum-Nickel-Cobalt), and SmCo (Samarium Cobalt). The magnetic forces within a magnetic force field follow the lines of magnetic flux. Magnetic forces are strongest where magnetic flux is most dense. Magnetic force fields penetrate most solids and liquids. A moving magnetic force field has two vectors: one in the direction of travel of the field and the other in the direction of the lines of magnetic flux.

Analyte Detection with Multiple Query Probes

In some embodiments, the technology relates to use of SiMREPS for detecting the presence, absence, and/or quantity of an analyte using query probes labeled with two or more different labels (e.g., fluorophores). In some embodiments, the technology comprises use of two or more query probes that are specific for the same analyte and that comprise two or more different labels.

In some embodiments, the two or more query probes comprise a first query probe comprising a first label and a second query probe comprising a second label (and, optionally, a third, fourth, fifth, sixth, seventh, eighth, ninth, tenth, etc. query probe comprising, respectively, a third, fourth, fifth, sixth, seventh, eighth, ninth, tenth, etc. label). In some embodiments, the first query probe is a different query probe than the second query probe (e.g., a composition comprises different query probes comprising different labels). In some embodiments, the first query probe is the same query probe as the second query probe (e.g., a first portion of the query probe molecules comprises a first label and a second portion of the query probe molecules comprises a second label). According to embodiments of the technology, the query probes comprising two or more different labels are provided in a composition for SiMREPS (e.g., an imaging buffer) and collected (e.g., deposited (e.g., immobilized)) on a surface as described herein. According to some embodiments, the surface-immobilized analyte is detected when both (or all) fluorophores repeatedly appear in the same location on the imaging surface (e.g., solid support), thus indicating the repeated binding of the multiple probes comprising each of the two or more labels.

In some embodiments, the two or more query probes comprise a first query probe comprising a first label and a second query probe comprising a second label and the first label and the second label are a Forster resonance energy transfer (FRET) pair. In some embodiments, the first query probe is a different query probe than the second query probe (e.g., a composition comprises different query probes comprising different labels that are a FRET pair). In some embodiments, the first query probe is the same query probe as the second query probe (e.g., a first portion of the query probe molecules comprises a first label and a second portion of the query probe molecules comprises a second label and the first label and the second label are a FRET pair). Accordingly, in some embodiments, query probes comprising labels that are a FRET pair bind to the same analyte simultaneously in a manner that positions the two FRET pair labels close enough that FRET occurs between the two labels (e.g., a distance closer than approximately the Førster radius of the two labels (e.g., fluorophores) (e.g., approximately 2-10 nanometers (e.g., 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 nm))) and the emission by the FRET acceptor is detected. Thus, the multiple (e.g., both) query probes are combined in a composition for SiMREPS (e.g., an imaging buffer) and surface-immobilized analyte is detected upon the repeated appearance of the FRET signal in the same location on the surface (e.g., the solid support).

In some embodiments using two or more labels (e.g., two or more fluorophores (e.g., a FRET pair)), detecting an analyte is associated with detecting (observing, recording, measuring) a signal (e.g., the repeated appearance of a signal or a signal feature) indicating a particular kinetic signature of switching between fluorescent and non-fluorescent states (or between FRET and non-FRET states). That is, in some embodiments, a particular kinetic signature indicates an increased confidence that the analyte is present. In both versions, the analyte is immobilized on a solid support (e.g., a coverslip, microscope slide, multiwell plate, diffusible particle (e.g., nanoparticle)) and/or immobilized to a fixed cell or other three-dimensional matrix prior to imaging in the presence of the query probes.

In some embodiments, use of two or more probes and/or two or more labels provides a more specific signal than use of a single fluorescent query probe. Accordingly, embodiments of the SiMREPS technology comprising use of two or more probes and/or two or more labels decreases the detection limit by reducing false positives. In some embodiments of the SiMREPS technology comprising use of two or more probes and/or two or more labels, the detection limit is not changed or not substantially changed and the acquisition time is shortened (e.g., by reducing the amount of time required to observe, record, and/or measure a kinetic signature of repeated binding of query probes to the analyte that is distinct from nonspecific binding of query probes to non-analyte entities. For example, under some circumstances, a single query probe may occasionally bind to the imaging surface and yield a signal that is similar to the signal provided by a repeatedly binding query probe to an analyte, but which is actually a signal produced by a photophysical process, e.g., repeated quenching and dequenching and/or repeated photoblinking (e.g., intersystem crossing between a dark triplet state and a fluorescing singlet state) rather than repeated binding. Thus, use of two or more query probes increases sensitivity and/or specificity because the likelihood of two differently labeled probes binding close to one another on a surface and producing a spurious repeated blinking signal is much lower than the likelihood of two differently labeled probes binding close together by binding the same analyte molecule.

### Assay Conditions for Modulating Query Probe Kinetics

In some embodiments, the technology relates to use of SiMREPS assay conditions that are provided to modulate (e.g., increase and/or decrease) the association of query probes to analytes and/or to modulate (e.g., increase and/or decrease) the dissociation of query probes from analytes. In some embodiments, modulating (e.g., increasing and/or decreasing) the association of query probes to analytes and/or modulating (e.g., increasing and/or decreasing) the dissociation of query probes from analytes results in modulating (e.g., increasing and/or decreasing) the assay time (e.g., time required to collect signals indicating the kinetic activity of query probe transient interactions with analytes). In particular embodiments, assay time is decreased by increasing the rate of query probe association with analytes and/or increasing the rate of query probe dissociation from analytes.

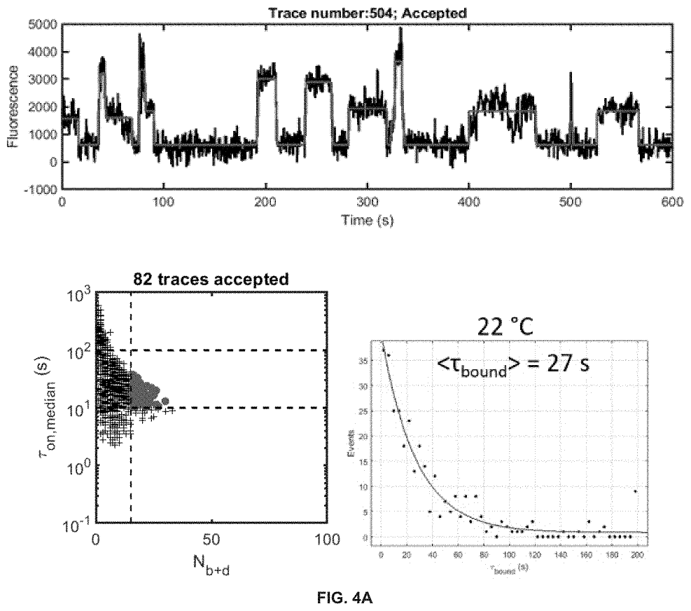

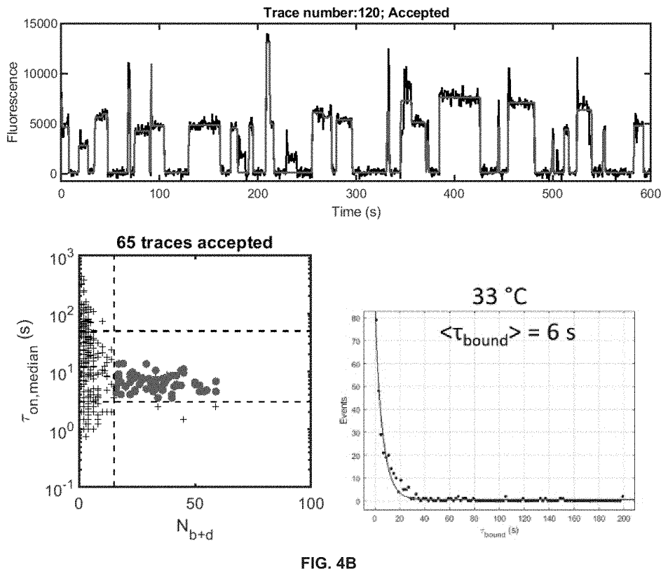

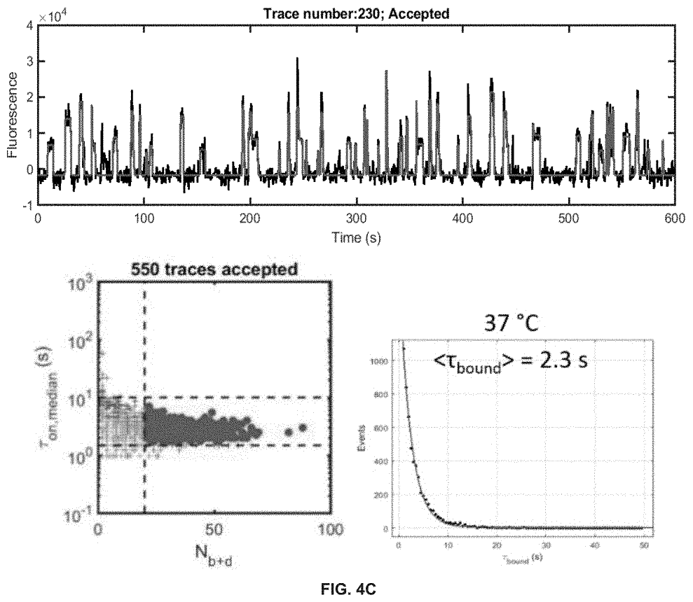

The technology includes various embodiments in which assay conditions are controlled to provide an improvement in the assay time. For example, in some embodiments, increasing the temperature at which SiMREPS assays are performed (e.g., using a thermocouple, microwave radiation, light, etc.) decreases the assay time, e.g., by increasing diffusion and weakening chemical interactions, thus increasing the rate of query probe association with the analyte and/or increasing the rate of query probe dissociation from the analyte (e.g., increasing query probe on/off rates). See FIG. 4A, FIG. 4B, and FIG. 4C. In some embodiments, the temperature is greater than 30° C. (e.g., greater than 30.0, 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, or 50.0° C.). In some embodiments, the temperature is maintained at a temperature between 30 to 50° C. (e.g., 30.5, 31.0, 31.5, 32.0, 32.5, 33.0, 33.5, 34.0, 34.5, 35.0, 35.5, 36.0, 36.5, 37.0, 37.5, 38.0, 38.5, 39.0, 39.5, 40.0, 40.5, 41.0, 41.5, 42.0, 42.5, 43.0, 43.5, 44.0, 44.5, 45.0, 45.5, 46.0, 46.5, 47.0, 47.5, 48.0, 48.5, 49.0, 49.5, or 50.0° C.) plus or minus 1-5° C. (e.g., ±1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0° C.).

In some embodiments, the technology comprises increasing the concentration of salt in a SiMREPS assay reaction mixture (e.g., imaging buffer) to weaken ionic interactions, thus increasing the rate of query probe association with the analyte and/or increasing the rate of query probe dissociation from the analyte (e.g., increasing query probe on/off rates). In some embodiments, the technology comprises increasing the concentration of organic solvents in a SiMREPS assay reaction mixture (e.g., imaging buffer) to weaken hydrophobic interactions, thus increasing the rate of query probe association with the analyte and/or increasing the rate of query probe dissociation from the analyte (e.g., increasing query probe on/off rates). In some embodiments, the salt concentration is increased to be more than 150 mM (e.g., 150 mM to 600 mM (e.g., 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 mM)). In some embodiments, the salt concentration is increased to be more than 100 mM (e.g., 100 mM to 1000 mM (e.g., 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, or 1000 mM)).

In some embodiments, the salt concentration is increased to be more than 150 mM monovalent (e.g., sodium) ions (e.g., 150 mM to 600 mM (e.g., 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 mM)). In some embodiments, the salt concentration is increased to be more than 100 mM monovalent (e.g., sodium) ions (e.g., 100 mM to 1000 mM (e.g., 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, or 1000 mM)).

In some embodiments, increasing the query probe on/off rate provides an increase in data collection rate and, accordingly, reduces the assay time and/or time needed for algorithms to identify (e.g., detect) analytes and discriminate analytes from background and false positive signals. In particular, the power of SiMREPS to distinguish between analytes and non-analytes increases as a function of the number of query probe binding events, e.g., SiMREPS discriminating power increases with a larger number of binding events of the query probe to a given molecule of the analyte. In other words, increasing the rate of association of the query probe with the analyte and/or dissociation of the query probe from the analyte, e.g., by manipulating salt concentration or temperature during the measurement, the rate of binding events per unit time increases (e.g., the same number of binding events can be observed in a shorter amount of time), thus providing the acquisition of a kinetic fingerprint sufficient to make a positive detection call for the analyte in a shorter period of time.

### Microfluidic Devices

In some embodiments, the technology relates to use of microfluidic sample handling for surface capture of an analyte followed by detection of the analyte by SiMREPS assay. In some embodiments, the technology (e.g., methods) comprises providing a microfluidic device comprising controlled channel dimensions; providing a sample comprising an analyte; and contacting the sample comprising the analyte in a microfluidic device comprising a small capture area coated with a capture probe (e.g., a capture antibody). In some embodiments, the technology maximizes the fraction (e.g., >5% (e.g., 5, 10, 15, 20, 25, 30, 35, 40, 45, 50, 55, 60, 65, 70, 75, 80, 85, 90, 91, 92, 93, 94, 95, 96, 97, 98, 99, 99.5%, or more)) of the analyte that is immobilized within the capture area.

In some embodiments, the technology comprises cyclically reloading fresh aliquots of the analyte sample or the same aliquot of the analyte sample into the device (see, e.g., Macdonald, Anal. Biochem., 2019, 566: 139-145, incorporated herein by reference). In these embodiments, fresh aliquots of the analyte sample are introduced into the microfluidic device at specified intervals (for example, at intervals of approximately every one to two minutes (e.g., at intervals of approximately 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 seconds)). Between the introduction of each aliquot of analyte sample, the capture area within the device is purged of the previous aliquot. Purging can be performed by washing the capture area with a buffer or other solution that does not comprise analyte or by pumping air (or another gas such as nitrogen) through the capture area within the device (also referred to as an air gap). Where air is used for purging, the purge time can be on the order of about one second (or less) or up to about 30-60 seconds (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 seconds). In some embodiments, the technology comprises cyclically reloading the same aliquot of the analyte sample into the device multiple times (also referred to as sample recycling). In these embodiments, an aliquot of the analyte sample is introduced into the microfluidic device and purged. Purging can be performed by washing the capture area with a buffer or other solution that does not contain analyte or by pumping air through the capture area within the device. After purging the capture area, the same aliquot of the analyte sample is then re-loaded into the capture area of the microfluidic device.

In some embodiments, the technology comprises controlling sample addition and mixing using digital microfluidic (DMF) approaches, wherein the manipulation of discrete droplets is electronically controlled (see, e.g., Miller, Anal Bioanal Chem, 2011, 339: 337-345; Shamsi, 2014, Lab Chip 14: 547-554, each of which is incorporated herein by reference).

In some embodiments, the technology comprises use of flow confinement for the concentration of analyte in the capture area within the microfluidic device (see, e.g., Hofmann, Anal. Chem. 2002, 74: 5243-5250, incorporated herein by reference). In these embodiments, a sample flow is joined with a confinement flow (e.g., water or sample medium). The confinement flow joins the sample flow in a perpendicular orientation. By using laminar flow conditions, no mixing occurs and, after confluence, a planar sheet geometry between the sample flow and confinement flow is obtained. Confinement of the sample can be controlled using the flow rate of the confinement flow.

In some embodiments, the technology comprises mixing the analyte sample within the microfluidic device, for example, within the capture area of the microfluidic device (see, e.g., Ward, 2015, J. Micromech Microeng, 25: 1-33, incorporated herein by reference). Microfluidic mixing can be separated into two categories: active and passive mixing. Passive mixing can be achieved by altering the structure or configuration of fluid channels and is incorporated into the system during fabrication. The extent of mixing is determined by the device configuration and is adjusted by using sample flow rates. Active mixers are activated and controlled by a user. Thus, some embodiments comprise passive mixing of the sample analyte within the capture area of the microfluidic device by introducing slanted wells, ridges, herringbone patterns, and/or grooves in the channel(s) of the microfluidic device or the analyte capture area of the device. In some embodiments, groove and/or ridge depth and/or height can be varied to affect mixing efficiency. In some embodiments, passive mixing is used with charged walls within the channels and/or capture area of the microfluidic device. For example, the substrate utilized to construct the device can have hydrophobic or charged characteristics.

Yet other embodiments comprise use of active mixing of the analyte sample within the capture area of the microfluidic device. In some embodiments, microstirrers are used to mix the analyte sample within the capture area of the microfluidic device. Some embodiments comprise use of acoustic waves to mix the analyte sample within the capture area of the microfluidic device. Acoustic waves can be combined with other mixing elements, such as microbubbles, to mix analyte samples in the capture area of a microfluidic device. Yet other embodiments comprise use of periodic fluid pulsation, thermal mixing, electrokinetic mixing, and/or other types of mixing of the analyte sample within the capture area of the microfluidic device. As would also be apparent to those skilled in the art, any combination of active and passive mixing can be used in the methods described herein.

In some embodiments, the sample is concentrated on the surface of a hydrogel material comprising immobilized capture molecules (e.g., using electrophoresis). In some embodiments, the hydrogel has a refractive index approximately 1.5 or greater and is compatible with total internal reflectance (see Zhou, 2013, Macromol Biosci 13: 1485-1491, incorporated herein by reference). In some instances, the hydrogel is molded into the shape of a prism or a rectangular slab.

Non-limiting additional examples of methods and techniques for concentrating and/or mixing analyte samples within the capture area of a microfluidic device are also disclosed in Glaser, 1993, Analytical Biochemistry 213: 152-161; Hibbert, 2002, Langmuir 18: 1770-1776; Gervais, 2006, Chemical Engineering Science 61: 1102-1121; Yang, 2008, Journal of Applied Physics 103: 084702-1-084702-10; Selmi, 2017, Scientific Reports 7: 1-11; Stott, 2010, PNAS 107: 18392-18397; Stroock, 2002, Science 295: 647-651; Green, 2007, Int. J. of Multiphysics 1: 1-32; Ward, 2015, J. Micromech Microeng. 25: 1-33; Hofmann, 2002, Analytical Chemistry 74: 5243-5250; and Macdonald, 2019, Analytical Chemistry 566: 139-145, each of which is hereby incorporated by reference in its entirety.

In contrast, data show that diffusion in a non-microfluidic sample cell (e.g., a cylindrical sample well affixed to the detection slide) provides an antigen capture efficiency of approximately 1% at the imaging surface. Controlled sample delivery to a small capture region with microfluidics is expected to yield a higher capture efficiency as well as capture over a smaller area, resulting in higher capture efficiency and sensitivity.

In some embodiments, the technology (e.g., methods) comprise contacting the captured analyte to an imaging solution comprising a query probe; and observing, recording, and/or measuring the transient, repeated association of the query probe with the analyte and dissociation of the query probe from the analyte. In some embodiments, the association and dissociation produces characteristic kinetics indicating the presence of the analyte in a discrete region of the solid support provided by the microfluidic device. In some embodiments, small channel dimensions provided by the microfluidic device improves the efficiency of analyte capture. In particular, a short diffusion distance (e.g., less than approximately 100 micrometers (e.g., less than 100, 90, 80, 70, 60, 50, 40, 30, 20, or 10 micrometers) increases the frequency of collisions between the analyte and the surface. In certain preferred embodiments, the channel dimension is such that the channel is 10 micrometers or less in depth (e.g., less than 9, 8, 7, 6, 5, 4, 3, 2, or 1 micrometers). Furthermore, use of a microfluidic device dramatically increases the effective concentration of the surface-bound capture probe in the adjacent section of the microfluidic channel due to the small cross-sectional area of the channel. Accordingly, use of a microfluidic device generally increases the kinetic rate of analyte capture by capture probes and drives the equilibrium towards the analyte-capture probe complex. The subsequent introduction of a reversibly binding query probe provides a SiMREPS assay for detecting the surface-bound antigen with high specificity and sensitivity by kinetic analysis of the signal arising from the association and dissociation of the query probe.

In various embodiments, microfluidic devices are fabricated from various materials using techniques such as laser stenciling, embossing, stamping, injection molding, masking, etching, and three-dimensional soft lithography. Laminated microfluidic devices are further fabricated with adhesive interlayers or by thermal adhesiveless bonding techniques, such as by pressure treatment of oriented polypropylene. The microarchitecture of laminated and molded microfluidic devices can differ. In some embodiments, the cartridge is generally fabricated using one or more of a variety of methods and materials suitable for microfabrication techniques. For example, in some embodiments the body of the device comprises a number of planar members that are individually injection molded parts fabricated from a variety of polymeric materials, or that are silicon, glass, or the like. In the case of crystalline substrates like silica, glass, or silicon, methods for etching, milling, drilling, etc. are used to produce wells and depressions that compose the various reaction chambers and fluid channels within the cartridge. Microfabrication techniques, such as those regularly used in the semiconductor and microelectronics industries, are particularly suited to these materials and methods. These techniques include, e.g., electrodeposition, low-pressure vapor deposition, photolithography (e.g., soft photolithography), etching, laser drilling, and the like. Where these methods are used, it will generally be desirable to fabricate the planar members of the device from materials similar to those used in the semiconductor industry, e.g., silica, silicon, or gallium arsenide substrates. U.S. Pat. No. 5,252,294, incorporated herein by reference in its entirety for all purposes, reports the fabrication of a silicon based multiwell apparatus for sample handling in biotechnology applications. In some embodiments, the microfluidic devices are prepared using multilayer soft lithography techniques. For example, in some embodiments of the technology relates, microfluidic devices are prepared as multilayer PDMS (e.g., Sylgard 183) devices (e.g., on a solid substrate, e.g., on glass) using multilayer soft lithographic techniques (MSL). See, e.g., Unger et al (2000) Science 288: 113-116 and International Patent Application WO2001001025, each incorporated herein by reference in its entirety.

In some embodiments, photolithographic methods of etching substrates are particularly well suited for the microfabrication of these microfluidic cartridges. For example, the first sheet of a substrate may be overlaid with a photoresist. An electromagnetic radiation source may then be shined through a photolithographic mask to expose the photoresist in a pattern that reflects the pattern of chambers and/or channels on the surface of the sheet. After removing the exposed photoresist, the exposed substrate may be etched to produce the desired wells and channels. Generally preferred photoresists include those used extensively in the semiconductor industry. Such materials include polymethyl methacrylate (PMMA) and its derivatives, and electron beam resists such as poly(olefin sulfones) and the like (more fully discussed in, e.g., Ghandi, “VLSI Fabrication Principles,” Wiley (1983) Chapter 10, incorporated herein by reference in its entirety for all purposes).

As used herein, the term “microfluidic channel” or “microchannel” refers to a fluid channel having variable length and one dimension in cross-section less than 500 to 1000 μm. Microfluidic fluid flow behavior in a microfluidic channel is highly non-ideal and laminar and may be more dependent on wall wetting properties, roughness, liquid viscosity, adhesion, and cohesion than on pressure drop from end to end or cross-sectional area. The microfluidic flow regime is often associated with the presence of “virtual liquid walls” in the channel. However, in larger channels, head pressures of 10 psi or more can generate transitional flow regimes bordering on turbulent, as can be important in rinse steps of assays.

In some embodiments, the microfluidic device comprises a pneumatic manifold that serves for control and fluid manipulation, although electronically activated valves find use in some embodiments. In some embodiments, air ports are connected to the pneumatic manifold. Air ports are provided in some embodiments with hydrophobic isolation filters (e.g., any liquid-impermeable, gas-permeable filter membrane) where leakage of fluid from within the device is undesirable and unsafe.

Some embodiments comprise a flexible membrane layer. The flexible membrane layer provides microfluidic valves and pumps. In some embodiments, the flexible membrane layer connects the cartridge to a controller deck where pressure and vacuum valves lie. The manipulation of the valves and pumps on the controller box applies either pressure or vacuum to the flexible membrane and moves the liquid through the channels by pneumatic actuation.

In some embodiments, reaction chambers are provided on the microfluidic device and can be any suitable shape, such as rectangular chambers, circular chambers, tapered chambers, serpentine channels, and various geometries for performing a reaction. These chambers may have observation windows (e.g., that allow the passage of electromagnetic radiation in the visible, ultraviolet, and/or infrared range of the spectrum), e.g., for examination of the contents (e.g., by a user, by a detector of a visible, ultraviolet, and/or infrared signal, etc.), e.g., to provide one or more detection chambers comprising a surface for a SiMREPS assay. Waste chambers are generally provided on the microfluidic devices. Waste chambers are optionally vented with sanitary hydrophobic membranes.

In some embodiments, the technology comprises non-microfluidic (e.g., macrofluidic) and microfluidic elements. In some embodiments, non-microfluidic elements (channels, chambers, etc.) are fluidically connected to each other and to microfluidic elements (channels, chambers, etc.) through microfluidic channels in a microfluidic device, e.g., a microfluidic device. The microfluidic device comprises directional control mechanisms, such as valves and pumps, by which fluid is selectively routed between different chambers and along different channels, and by which a single chamber can communicate with a number of other chambers. These connections and routing mechanisms allow automation of functions performed by the microfluidic device.

Covalent Association of Analyte with Imaging Surface

In some embodiments, the technology relates to covalently linking an analyte to a SiMREPS imaging surface (e.g., by formation of a chemical bond between the analyte and the surface and/or between the analyte and a capture probe immobilized to the surface). In some embodiments, the technology increases the sensitivity of detecting an analyte by SiMREPS. In particular, while SiMREPS and other surface-based assays comprise use of a capture probe to immobilize the analyte of interest (e.g., an antigen) to a surface for detection, the affinity of the capture probe for the analyte is finite and for many analyte-capture probe pairs the fraction of analyte that dissociates from the surface is significant (e.g., greater than 10%) on a timescale of minutes or hours. As a result, the amount of analyte on the surface decreases over time, resulting in lower sensitivity and potentially lower reproducibility if the time interval between capture and detection is not well-controlled. For example, data were collected that indicate that the sensitivity of different SiMREPS assays of proteins decreases over time, which strongly suggests that the dissociation of the antigen from the surface is significant.

Accordingly, in some embodiments, the SiMREPS technology provided herein reduces analyte dissociation to improve the sensitivity of SiMREPS and other surface-based measurements. In particular, embodiments of the technology provided herein provide one or more covalent bonds that cross-link the analyte to a capture probe, thus preventing dissociation of the analyte from the surface prior to or during the measurements. The technology is not limited in the chemistry use to produce a cross-link between an analyte and a capture probe. For example, embodiments incubating the analyte-capture probe complex with a reactive chemical such as an NHS ester derivative (e.g., disuccinimidyl tartrate, disuccinimidyl suberate, or disuccinimidyl glutarate), imidoester derivative (e.g., dimethyl pimelimidate, dimethyl suberimidate), haloacetyl derivative (e.g., succinimidyl iodoacetate), maleimide derivative (e.g., succinimidyl 4-(N-maleimidomethyl)cyclohexane-1-carboxylate), or carbodiimide derivative (e.g., 1-ethyl-3-(3-dimethylaminopropyl) carbodiimide); or by irradiation of the analyte-capture probe complex with UV light. After cross-liking the analyte to the capture probe, the captured analyte is detected by SiMREPS, e.g., using imaging in the presence of a or query probe that transiently binds to the captured analyte with characteristic kinetics constituting a kinetic fingerprint that can be detected, e.g., by total internal reflection fluorescence microscopy. In some embodiments, it is important that the cross-linking agent does interfere substantially with the interaction between the query probe and analyte. For example, it is preferable if the region of the analyte (e.g., epitope) with which the query probe interacts is free of the functional groups involved in cross-linking.

### EXAMPLES

### Example 1

During the development of embodiments of the technology provided herein, experiments were conducted to test gold nanoparticles having a diameter of approximately 100 nm and comprising a capture probe and to test magnetic nanoparticles having a diameter of approximately 100 nm and comprising a capture probe to capture analytes and subsequent SiMREPS analysis as described herein. In these experiments, data were collected indicating that use of nanoparticles comprising capture probes is compatible with SiMREPS detection of both nucleic acid and protein analytes.

### Example 2

During the development of embodiments of the technology provided herein, experiments were conducted to modulate SiMREPS assay acquisition time by providing an increased temperature and/or salt concentration. Data collected during the experiments indicated that the speed of SiMREPS kinetic fingerprinting assays using query probes comprising fluorescently labeled monovalent Fab fragments can be increased by over 10-fold by manipulating salt and/or temperature, thus reducing SiMREPS assay time by 90% or more in some embodiments.

For example, data collected during these experiments indicated that increasing the SiMREPS assay temperature to 30-37° C. provides an improved SiMREPS assay of an interleukin-6 (IL-6) antigen analyte using a query probe comprising an antibody (fluorescent Fab fragment) that interacts transiently with the IL-6 antigen (e.g., analyte). By increasing the assay temperature to 33° C. using a heated stage or to approximately 33-37° C. using an objective lens heater, SiMREPS specifically detected the IL-6 antigen analyte in approximately 2 minutes. In contrast, specific detection of the IL-6 antigen by SiMREPS at room temperature (e.g., approximately 22-24° C.) occurred in 20 minutes. Similarly, data collected during these experiments indicated that a SiMREPS assay to detect plasminogen activator inhibitor-1 (PAI-1) protein was completed in approximately 2 minutes at an assay temperature of approximately 30-37° C. In contrast, a SiMREPS assay to detect PAI-1 protein at room temperature occurred in approximately 10 minutes. Accordingly, these data indicate that increasing the temperature of SiMREPS assays provides a general method to increase the acquisition rate of SiMREPS measurements with dynamically binding and dissociating query probes. See FIG. 4.

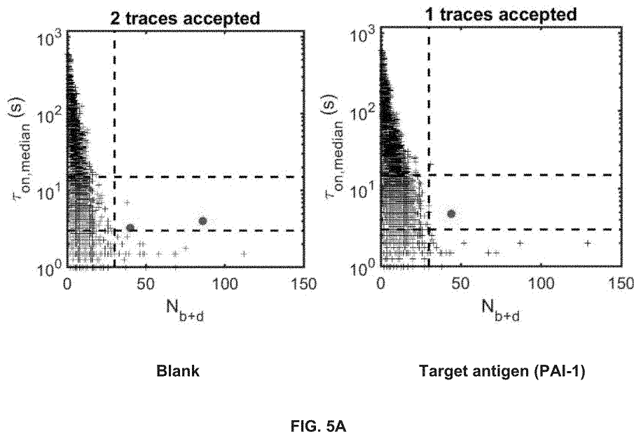

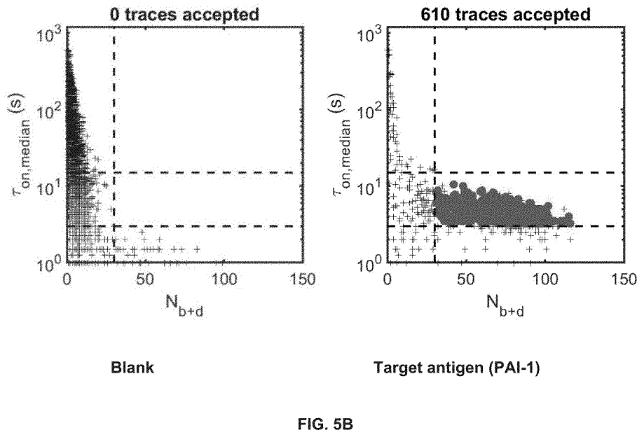

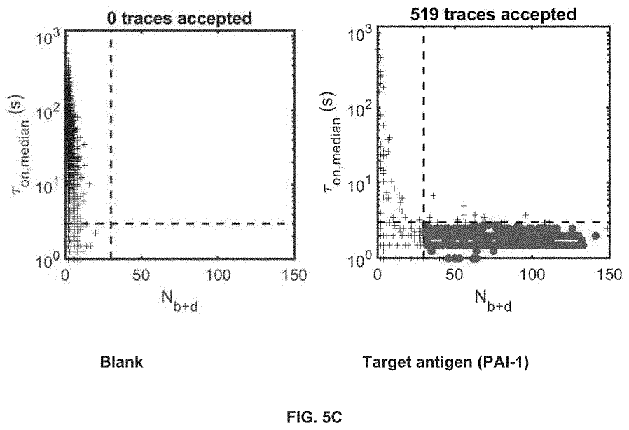

In addition, data collected during experiments indicated that increasing the salt concentration of the SiMREPS reaction mixture (e.g., imaging buffer) from approximately 150 mM sodium ions to approximately 600 mM sodium ions accelerates the acquisition of data for a SiMREPS assay to detect PAI-1 analyte using a query probe comprising a fluorescent Fab fragment by a factor of approximately 5: e.g., from 10 minutes to 2 minutes per field of view. Without being bound by theory, increasing the concentration of sodium ions decreases the residence time of the query probe binding to the analyte and decreases the frequency of nonspecific binding of the query probe to the detection surface. See FIG. 5.

Accordingly, the data indicate that increasing the temperature and/or the salt concentration improve the power of SiMREPS to distinguish the kinetic fingerprint of query probe binding to the analyte from background kinetics in a shorter period of time.

### Example 3

During the development of embodiments of the technology provided herein, experiments were conducted to quantify four different protein antigens using an embodiment of the SiMREPS technology described herein. In particular, a series of standard curves was produced for PAI-1, IL-6, VEGF-A, and IL-34, which indicated quantitative detection of these four protein analytes using SiMREPS kinetic fingerprinting with fluorescently labeled query probes. FIG. 6.

The matrix was animal serum (horse serum for PAI-1 and IL-6; chicken serum for VEGF-A and IL-34). Apparent limits of detection were 770 aM for PAI-1, 770 aM for IL-6, 3.6 fM for VEGF-A, and 6.5 fM for IL-34, which were calculated as three standard deviations above the mean of the blank. The data indicated that between 250 and 1300 molecules were captured on the imaging surface per femtomolar of antigen in the 100-microliter samples, corresponding to a capture efficiency of 0.4-2.2% for these particular experimental conditions.

### Example 4

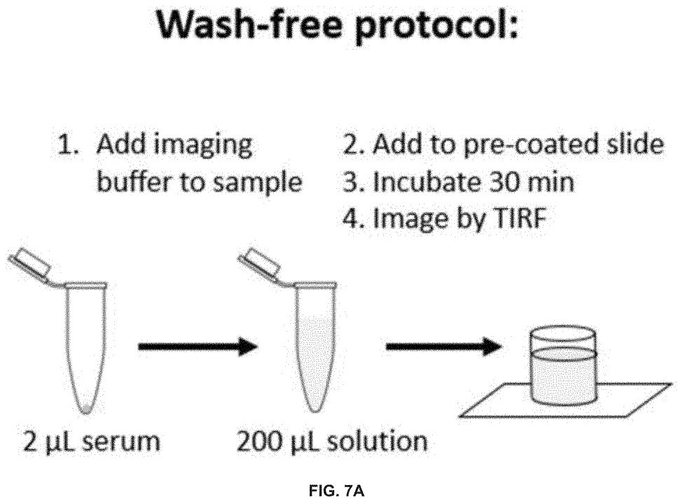

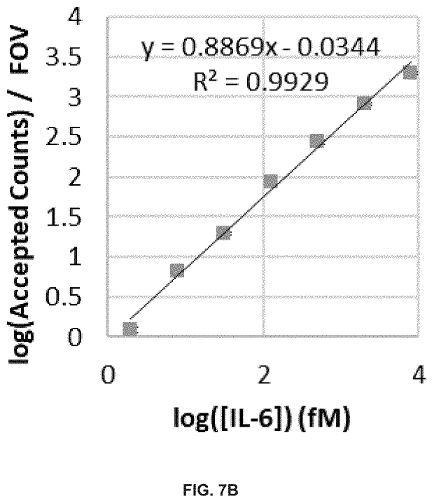

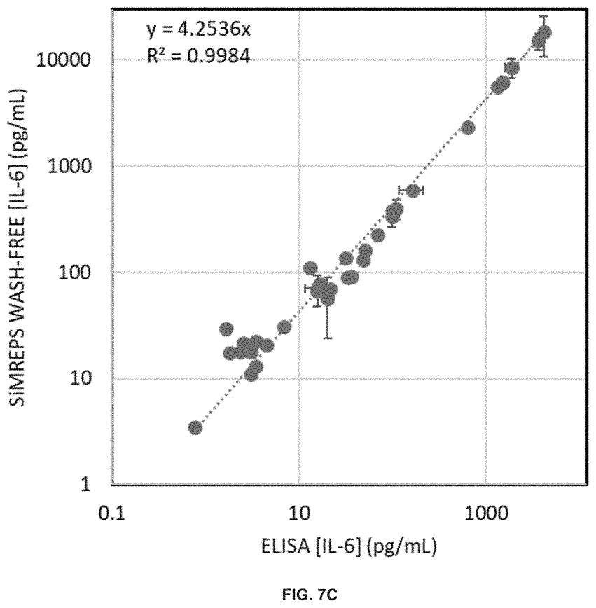

During the development of embodiments of the technology provided herein, experiments were conducted to develop and test a wash-free protocol for SiMREPS. FIG. 7A. The protocol was used for SiMREPS and provided quantitative detection of IL-6 in serum. In this protocol, the serum sample containing IL-6 was combined with the imaging solution comprising the query probe and then added to a coverslip that was pre-coated with a capture antibody. After incubation (e.g. 30 minutes), the sample was imaged by TIRF microscopy to quantify IL-6. A standard curve was produced using data from kinetic fingerprinting of IL-6 with the wash-free protocol. FIG. 7B. A correlation plot was produced using data from measuring IL-6 in 34 patient-derived (human) serum samples by SiMREPS (no-wash protocol, 100-fold dilution for all samples) and ELISA (variable dilution factors, 4- or 64-fold, depending on analyte concentration). The correlation coefficient between the two methods was 0.999. FIG. 7C. In contrast to ELISA, the SiMREPS protocol avoids washing steps following sample introduction. In addition, SiMREPS provides an improved method for detecting analytes (e.g., protein analytes) relative to ELISA because SiMREPS uses samples that are up to 25-fold more dilute than samples for ELISA.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A system for detecting an analyte, said system comprising:
a capture probe that stably binds the analyte;
a query probe that transiently binds to the analyte; and
a microfluidic device comprising a substrate and a capture area in which the capture probe is immobilized.

2. The system of claim 1, wherein the capture probe comprises an antibody.

3. The system of claim 1, wherein the query probe comprises a nucleic acid, the capture probe comprises a nucleic acid, and/or the analyte comprises a nucleic acid.

4. The system of claim 1, wherein the query probe comprises an antigen-binding antibody fragment, a monovalent Fab, a nanobody, a single-chain variable fragment antibody, an aptamer, or an antibody.

5. The system of claim 1, wherein the query probe comprises a label.

6. The system of claim 1, wherein the query probe comprises a fluorescent label.

7. The system of claim 1, wherein the substrate is a substantially planar surface.

8. The system of claim 1, further comprising a detection component to detect transient binding of the query probe to the analyte.

9. The system of claim 1, further comprising a computer configured to receive and analyze kinetic data describing the association of the query probe with the protein analyte and dissociation of the query probe from the protein analyte.

10. The system of claim 1, wherein the analyte is mixed in the capture area of the microfluidic device.

11. The system of claim 10, wherein the analyte is mixed by active and/or passive mixing systems.

12. The system of claim 11, wherein the active and/or passive mixing systems are selected from microstirrers, acoustic waves, microbubbles, periodic fluid pulsation, thermal mixing, electrokinetic mixing, ridges in the microfluidic device channel and/or capture area, herringbone structures in the microfluidic device channel and/or capture area, and combinations thereof.

13-21. (canceled)

22. The system of claim 1, said system further comprising a temperature-control component configured to maintain the microfluidic device at about 25 to about 50° C.

23-26. (canceled)

27. The system of claim 1, said system further comprising one or more component that concentrates the analyte.

28. The system of claim 27, said component providing electrophoretic stacking, electrophoretic focusing, flow confinement, cyclical reloading of analyte sample, and/or temperature gradient focusing of said analyte.

29. (canceled)

30. (canceled)

31. A method comprising:
providing a system comprising:
a capture probe that stably binds an analyte;
a query probe that transiently binds to the analyte; and
a microfluidic device comprising a substrate and a capture area in which the capture probe is immobilized; and
detecting and/or quantifying the analyte in said sample.

32. (canceled)

33. The method of claim 31, wherein said sample is a biofluid.

34. (canceled)

35. (canceled)

36. The method of claim 31, wherein said analyte comprises a protein, nucleic acid, or metabolite.

37. The method of claim 31, further comprising providing a result describing the presence and/or quantity of said analyte in said sample.

38. (canceled)

39. The method of claim 31, further comprising providing a standard curve.

40-78. (canceled)
